(19) United States
(12) Patent Application Publication
(10) Pub. No.: US 2025/0320172 A1
(43) Pub. Date: Oct. 16, 2025

(54) MILD ELECTROCHEMICAL DECARBOXYLATIVE ALKYL-ALKYL COUPLING AND DECARBOXLATIVE OLEFINATION ENABLED BY RAPID ALTERNATING POLARITY

(71) Applicant: THE SCRIPPS RESEARCH INSTITUTE, La Jolla, CA (US)

(72) Inventors: Yu KAWAMATA, San Diego, CA (US); Yuta HIOKI, Tokyo (JP); Phil S. BARAN, San Diego, CA (US)

(21) Appl. No.: 18/868,825

(22) PCT Filed: Jun. 14, 2023
(86) PCT No.: PCT/US2023/068403

§ 371 Date: Nov. 25, 2024

Related U.S. Application Data
(60) Provisional application No. 63/352,091, filed on Jun. 14, 2022.

(51) Int. Cl. C07C 1/207; C07C 29/147; C07C 227/18

(52) U.S. Cl. CPC C07C 1/2078; C07C 29/147; C07C 227/18

(57) ABSTRACT

Rapid Alternating Polarity (rAP) is a new electrolysis mode for synthetic organic electrochemistry. As described herein, AC waveforms, particularly rAP, can profoundly alter the reaction outcome of the reduction of carbonyl groups and arenes, exhibiting unprecedented levels of chemoselectivity that is absent when DC is used under otherwise identical reaction conditions. Herein, disclosed are new applications of rAP electrolysis, such as i) rAP-Kolbe electrolysis for the decarboxylative coupling of carboxylic acids; and ii) rAP electrolysis for the decarboxylative olefination of carboxylic acids; both under mild electrochemical conditions.

### CROSS-REFERENCE

This application claims the benefit of U.S. Provisional Application No. 63/352,091, filed on Jun. 14, 2022, which is incorporated herein by reference in its entirety.

### GOVERNMENT SUPPORT

This invention was made with government support under grant number GM-118176 awarded by the National Institutes of Health and grant number CHE-2002158 awarded by the National Science Foundation.

### FIELD OF THE INVENTION

Rapid Alternating Polarity (rAP) is a new electrolysis mode for synthetic organic electrochemistry. As described herein, AC waveforms, particularly rAP, can profoundly alter the reaction outcome of the reduction of carbonyl groups and arenes, exhibiting unprecedented levels of chemoselectivity that is absent when DC is used under otherwise identical reaction conditions. Herein, disclosed are new applications of rAP electrolysis, such as i) rAP-Kolbe electrolysis for the decarboxylative coupling of carboxylic acids; and ii) rAP electrolysis for the decarboxylative olefination of carboxylic acids; both under mild electrochemical conditions.

### BACKGROUND OF THE INVENTION

The Kolbe electrolysis has been extensively studied since its first appearance in literature in the mid-19th century. In general, oxidative decarboxylation of an aliphatic carboxylic acid generates a transient alkyl radical, which combines to form a Csp³-Csp³ bond via radical-radical coupling. Such a transformation is tremendously useful due to the wide availability of carboxylic acids and necessity of no exogenous costly additives to facilitate decarboxylation. It shortens synthesis by avoiding excessive protecting/redox manipulations typically associated with conventional Csp³-Csp³ bond formation tactics such as Grignard addition or Wittig-type olefinations followed by hydrogenation. Despite these attractive features, this reaction has had little success in practical synthesis due to the poor functional group compatibility resulting from the extremely high overpotentials employed and the requirement of expensive Pt electrodes. The low chemoselectivity can be directly traced to the harsh electrolysis conditions employed, which severely limit the menu of compatible functional groups (only esters and ethers).

Even compatibility with a seemingly inert terminal olefin is problematic as discussed below. In addition to this limited functional group compatibility, Pt anode is considered essential for performing the reaction. This is commonly explained by the unique property of Pt surface to adsorb carboxylates to facilitate its oxidation over solvent oxidation. Although alternative inexpensive anode materials are a subject of active research, no practical solution has yet been found.

Rapid Alternating Polarity (rAP) is a new electrolysis mode for synthetic organic electrochemistry. Traditionally, direct current was applied to electrochemical synthesis of organic compounds, where electricity is applied by holding either current (I) or potential (V) at a constant value (FIG. 1, left). Although much less common, alternating current instead of direct current can be applied to electrochemical synthesis as well. Alternating current refers to electric current that periodically changes its direction. For example, the potential change of alternating current follows a sine-wave pattern (FIG. 1, middle) as well as square waveform (FIG. 1, right). Square waveform can be easily accessed by simply alternating the electrode polarity; thus, we refer this waveform as rapid Alternating Polarity (rAP) to distinguish it from other types of AC waveforms. Although, application of AC waveforms into organic synthesis has received little attention, rAP has recently been found to exhibit unique chemiselectivity and reactivity when it is applied to electroreductive transformations. In contrast, application of rAP into electrooxidative transformation has been underexplored.

Further exploration of these unexpected effects led to the discovery, as disclosed herein, of new applications of rAP into Kolbe electrolysis which is one of the oldest, yet underutilized, electrochemical reactions known to date. Herein disclosed are new applications of the introduction of rAP into Kolbe electrolysis. This new rAP-Kolbe electrolysis technology has been used to address the current need in the field for more efficient and less costly methods for the decarboxylation of carboxylic acids. Additionally, rAP electrolysis technology, as also demonstrated herein, has been used to conduct efficient and cost-effective methods of the decarboxylative olefination of carboxylic acids.

### BRIEF DESCRIPTION OF THE FIGURES

FIG. 1. Waveform of direct current, typical alternating current and rAP.

FIG. 2. rAP-Kolbe dimerization of 10-undecanoic acid 1 and comparisons with Pt electrodes and DC current.

FIG. 3. Scope of rAP-Kolbe dimerization.

FIG. 4. Scope of rAP-Kolbe heterocoupling.

FIG. 5. Scope of rAP-decarboxylative olefination.

FIG. 6. Application examples of the rAP-Kolbe products.

### SUMMARY OF THE INVENTION

The application provides rapid Alternating Polarity (rAP) which is a new mode of electrolysis for synthetic organic chemistry methods. Specifically disclosed herein are methods of applying rAP modes to Kolbe electrolysis. The application further provides methods of decarboxylative olefination of carboxylic acids using this new rAP electrolysis technology.

Disclosed herein is the discovery that Kolbe electrolysis can proceed on carbon-based electrodes by applying rAP rather than DC, enabling the coupling of carboxylic acids that are challenging with conventional Pt electrodes. This new process is referred to herein as rAP-Kolbe electrolysis. The rAP-Kolbe electrolysis technology has been used to address the current need in the field for more efficient and less costly methods for the decarboxylation of carboxylic acids. Similarly, rAP-Kolbe electrolysis technology, as also demonstrated herein, has been used to conduct efficient and cost-effective methods of the decarboxylative olefination of carboxylic acids.

The application herein provides a method of decarboxylative coupling of carboxylic acids, wherein a carboxylic acid substrate is subjected to rapid Alternating Polarity (rAP)-Kolbe electrolysis.

The application herein provides the above method, wherein the rAP-Kolbe electrolysis occurs according to either reaction scheme (i) or reaction scheme (ii):

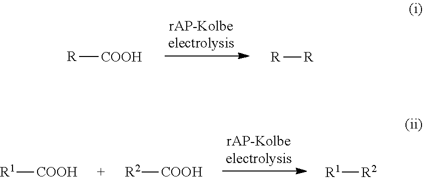

wherein each of R, R¹, and R² is independently selected from H, saturated or unsaturated acyclic or cyclic aliphatic groups, each optionally including one or more heteroatoms and each optionally substituted with alkyl, alkenyl, ester, amino, halo, hydroxy, keto, formyl, aryl, heteroaryl, cycloalkyl or heterocyclic moieties, and aromatic or heteroaromatic ring systems, each optionally substituted with alkyl, alkenyl, ester, amino, halo, hydroxy, keto, formyl, aryl, heteroaryl, cycloalkyl or heterocyclic moieties.

The application further provides the above methods of decarboxylative coupling of carboxylic acids, wherein the carboxylic substrate comprises one or two amino acids or derivatives thereof.

The application herein provides a method of decarboxylative olefination of carboxylic acids, wherein the carboxylic acid substrate is subjected to rAP electrolysis.

The application herein provides the above method of decarboxylative olefination of carboxylic acids, wherein the rAP electrolysis occurs according to either reaction scheme (i) or reaction scheme (ii):

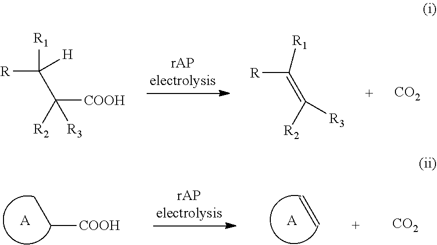

wherein R¹, R² and R³ are optionally H or substituted (C₁-C₂₀)alkyl, (C₂-C₂₀)alkenyl, (C₁-C₂₀)hydroxyalkyl, and N-protected (C₁-C₂₀)aminoalkyl; and
A is optionally substituted, saturated or partially unsaturated, (C₃-C₈)cycloalkyl or (C₃-C₈)heterocycloalkyl.

### DETAILED DESCRIPTION OF THE INVENTION

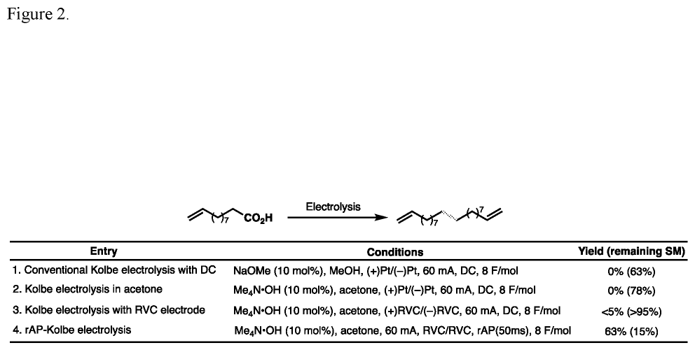

Herein demonstrated is the effect of rAP on Kolbe electrolysis. For example, the dimerization of 10-undecenoic acid has been achieved using rAP-Kolbe electrolysis as shown in FIG. 2. Indeed, the dimerization of such a simple carboxylic acid was inoperable under conventional Kolbe electrolysis conditions with a Pt anode (FIG. 2, entry 1). In fact, this is consistent with the literature report that Kolbe dimerization of this substrate yielded no desired coupling product (Compound 2, FIG. 2) due to extensive electrode passivation. The experiment with Pt electrodes was repeated in acetone, also to no avail (FIG. 2, entry 2). Switching electrodes into RVC electrodes had no improvement (FIG. 2, entry 3) with DC current. Surprisingly, the dimerization proceeded with high efficiency by simply switching the current delivery to rAP, furnishing the desired dimer in 63% yield (FIG. 2, entry 4). Although Kolbe electrolysis has been investigated with AC current, such a dramatic effect has not been reported to date. This rAP-Kolbe reaction enables the dimerization of various carboxylic acids that are traditionally challenging, offering highly economical access to many compounds of commercial interest.

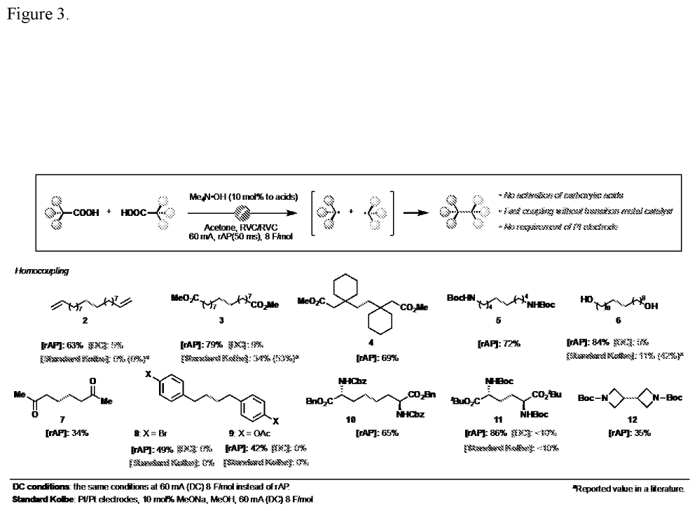

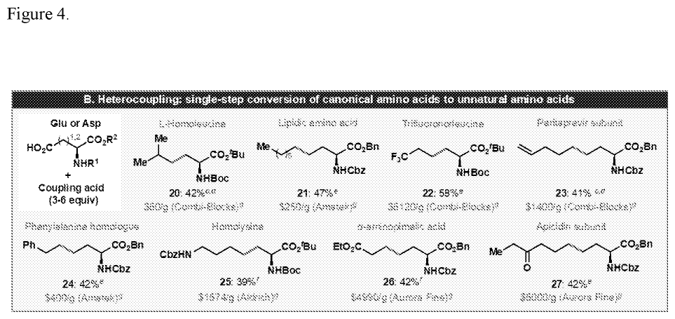

FIG. 3 demonstrates the scope of the rAP-Kolbe dimerization. The functional group tolerance was greatly expanded with this new method, and now various acids containing ester (3), amino (5), free hydroxy (6), ketone (7) and even aryl groups that are susceptible for oxidative degradation (8, 9) afford the corresponding dimer in a good yield. The method can be applied to dimerizing amino acids to synthesize high-value unnatural amino acid without losing chiral information (10 and 11 was obtained as a single diastereomer). Even azetidine 3-carboxylic acid can be dimerized in synthetically useful yield (12). Conventional Kolbe electrolysis with Pt electrode as well as DC electrolysis under the same conditions to rAP gave little or no desired dimers in most cases. Several compounds in FIG. 3 are known to have commercial value. For example, diester 3, diamine 5 and diol 6 have found applications in polymers, cosmetics and lubricant sectors. Notably, possessing long-chain alkyl groups makes these compounds attractive starting materials for degradable polyethylene alternatives, which is of great importance to improve recyclability of commodity polymers. Prior routes to such compounds rely on expensive Ru-based catalysis. Compound 10 and 11 are valuable unnatural amino acids, and have been used in drug discovery. Particularly, 10 is frequently used as more stable surrogate of disulfide linkage due to the structural similarity to cystine. Additionally, rAP-cross Kolbe is also possible (FIG. 4). Nonetheless, high-value unnatural amino acids can be directly obtained from readily available glutamic acid or aspartic acid in a single step without necessitating any expensive reagent or catalyst.

Overall, establishing Csp³-Csp³ bond at a position remote from a functional group is challenging in general due to the lack of synthetic handle. Multi-step protecting/redox manipulations and the requirement of expensive reagents or catalysts are nearly unavoidable in conventional strategies for making such bonds. In this context, Kolbe electrolysis represents one of the most straightforward (i.e. most economical) methods due to the simplicity of the reaction conditions and starting material availability. FIGS. 3 and 4 clearly illustrate that rAP-Kolbe electrolysis represents a breakthrough in this regard.

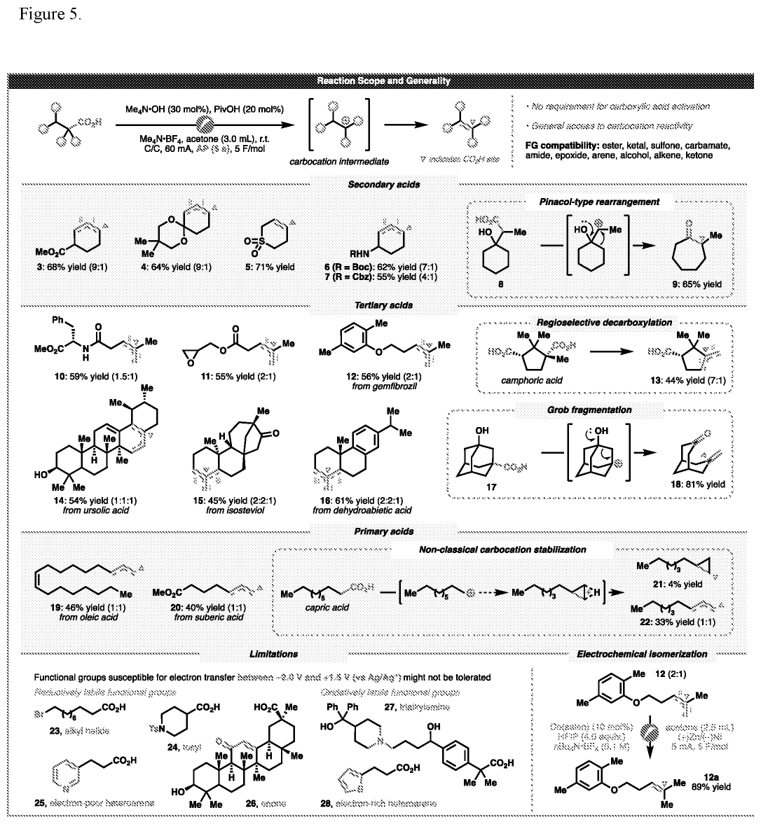

FIG. 5 demonstrates the scope of the decarboxylative olefination. Decarboxylative olefination proceeded under rAP electrolysis with modified conditions. Several carboxylic acid substrates with functional groups (such as amino, free hydroxy, ester) afforded corresponding alkenyl products.

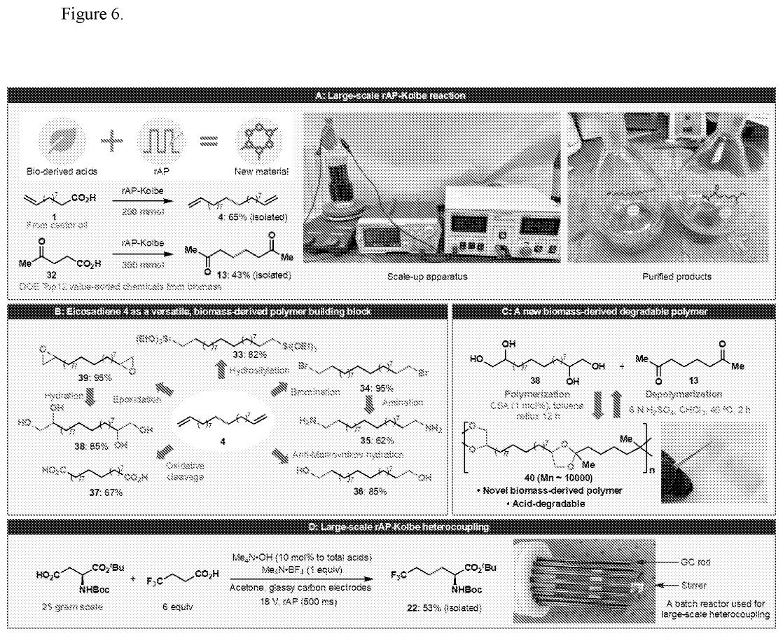

FIG. 6 demonstrates the examples of the application of the rAP-Kolbe products. This method enables access to several compounds that have high commercial value or potential. Moreover, rAP-Kolbe electrolysis could hold great promise from the viewpoint of biomass conversion, since carboxylic acids are ubiquitously found in biomass. In fact, 10-undecenoic acid (starting material for diene 2) is a biomass-derived carboxylic acid; rAP-Kolbe electrolysis has potential for commercial production of sophisticated polymer building blocks from biomass. Furthermore, amino acids and their derivatives are one of the most versatile chiral building blocks for pharmaceuticals and other chemicals. These compounds are clearly outside the reach of conventional Kolbe electrolysis as demonstrated in FIG. 2.

### REFERENCES

### Representative Review for Kolbe Electrolysis

1. Schafer, H.-J. Electrochemistry IV. Top. Curr. Chem. 91-151 (Springer 2005).
2. Schafer, H. J. Recent synthetic applications of the Kolbe electrolysis. Chem. Phys. Lipids 24, 321-333 (1979).

### Patents Using Kolbe Electrolysis for Industrial Applications:

1. Isoya, T.; Kakuta, R.; Kawamura, C. Process for the Preparation of Sebacic acid. U.S. Pat. No. 3,896,011, Jul. 22, 1975.
2. Publications on the use of rAP-based methods for reductive processes (the current process is oxidative):
3. Kawamata, Y.; Hayashi, K.; Carlson, E.; Shaji, S.; Waldmann, D.; Simmons, B. J.; Edwards, J. T.; Zapf, C. W.; Saito, M.; Baran, P. S. Chemoselective Electrosynthesis Using Rapid Alternating Polarity. J. Am. Chem. Soc. 2021, 143, 16580-16588.
4. Hayashi, K.; Griffin, J.; Harper, K. C.; Kawamata, Y.; Baran, P. S. Chemoselective (Hetero)Arene Electroreduction Enabled by Rapid Alternating Polarity, J. Am. Chem. Soc. 2022, 144, 5762-5768.

### Patents, Publications and Public Use of Some of the Materials Outlined Above:

5. Häußler, M.; Eck, M.; Rothauer, D.; Mecking, S. Closed-loop recycling of polyethylene-like materials, Nature, 2021, 590, 423-427.
6. https://elevance.com/wp-content/uploads/2017/09/Elevance-ODDA-C18-white-paper_20130916_F.pdf
7. Chinese Patent for the production of C18 diacid: CN202111676791 20211231.
8. Blaskovich, M. A. T. Unnatural Amino Acids in Medicinal Chemistry, J. Med. Chem. 2016, 59, 10807-10836.
9. Goudreau, N.; Brochu, C.; Cameron, d. R.; Duceppe, J.-S.; Faucher, A.-M.; Ferland, J.-M.; Grand-Maitre; Poirier, M.; Simoneau, B.; Tsantrizos, Y. S. Potent inhibitors of the hepatitis C virus NS3 protease: design and synthesis of macrocyclic substrate-based β-strand mimics. J. Org. Chem. 2004, 69, 6185.

### EMBODIMENTS

The application provides the following embodiments:

Embodiment 1. A method of decarboxylative coupling of carboxylic acids, wherein a carboxylic acid substrate is subjected to rapid Alternating Polarity (rAP)-Kolbe electrolysis.

Embodiment 2. The method of Embodiment 1, wherein the rAP-Kolbe electrolysis occurs according to either reaction scheme (i) or reaction scheme (ii):

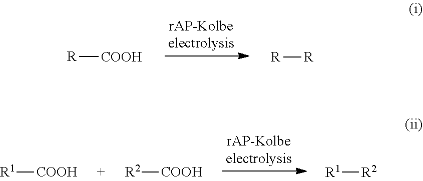

wherein each of R, R¹, and R² is independently selected from H, saturated or unsaturated acyclic or cyclic aliphatic groups, each optionally including one or more heteroatoms and each optionally substituted with alkyl, alkenyl, ester, amino, halo, hydroxy, keto, formyl, aryl, heteroaryl, cycloalkyl or heterocyclic moieties, and aromatic or heteroaromatic ring systems, each optionally substituted with alkyl, alkenyl, ester, amino, halo, hydroxy, keto, formyl, aryl, heteroaryl, cycloalkyl or heterocyclic moieties.

Embodiment 3. The method of decarboxylative coupling of carboxylic acids of either Embodiment 1 or Embodiment 2, wherein the alternating frequency is 1 MHz-0.01 Hz.

Embodiment 4. The method of decarboxylative coupling of carboxylic acids of any one of Embodiments 1-3, wherein the electrodes are composed of Pt, Pd, Ni, Rh, Ti, Pb and other metals, conductive metal oxides, or a carbon-based material including surface-modified materials.

Embodiment 5. The method of decarboxylative coupling of carboxylic acids of any one of Embodiments 1-4, wherein the carboxylic acid substrate contains 2-100 carbon atoms.

Embodiment 6. The method of decarboxylative coupling of carboxylic acids of any one of Embodiments 1-5, wherein the carboxylic acid substrate contains heteroatoms.

Embodiment 7. The method of decarboxylative coupling of carboxylic acids of any one of Embodiments 1-6, wherein the carboxylic acid substrate is unsaturated, partially unsaturated, or aromatic.

Embodiment 8. The method of decarboxylative coupling of carboxylic acids of any one of Embodiments 1-7, wherein the carboxylic acid substrate contains a C—N bond.

Embodiment 9. The method of decarboxylative coupling of carboxylic acids of any one of Embodiments 1-8, wherein the carboxylic acid substrate contains alkenyl, ester, amino, free hydroxy, cycloalkyl, ketone, aryl, or heterocycloalkyl moieties.

Embodiment 10. The method of decarboxylative coupling of carboxylic acids of any one of Embodiments 1-9, wherein a supporting electrolyte is added.

Embodiment 11. The method of decarboxylative coupling of carboxylic acids of any one of Embodiments 1-10, wherein the supporting electrolyte is an ammonium salt.

Embodiment 12. The method of decarboxylative coupling of carboxylic acids of any one of Embodiments 1-11, wherein the decarboxylative coupling of carboxylic acids occurs in the presence of a base.

Embodiment 13. The method of decarboxylative coupling of carboxylic acids of Embodiment 12, wherein the amount of base is approximately 0.01 mol % to approximately 200 mol % to the overall amount of the carboxylic acid substrate.

Embodiment 14. The method of decarboxylative coupling of carboxylic acids of Embodiment 13, wherein the amount of the base is approximately 10 to approximately 30 mol % to the overall amount of the carboxylic acid substrate.

Embodiment 15. The method of decarboxylative coupling of carboxylic acids of any one of Embodiments 12-14, wherein the base is NR₄OH, NH₄OH, NaOH, or KOH and R is (C₁-C₆)alkyl.

Embodiment 16. The method of decarboxylative coupling of carboxylic acids of Embodiment 15, wherein the base is NR₄OH and R is (C₁-C₆)alkyl.

Embodiment 17. The method of decarboxylative coupling of carboxylic acids of Embodiment 16, wherein the amount of NR₄OH is approximately 10 mol % to the overall amount of the carboxylic acid substrate.

Embodiment 18. The method of decarboxylative coupling of carboxylic acids of any one of Embodiments 1-17, wherein the solvent has a relative dielectric constant of more than 5.

Embodiment 19. The method of decarboxylative coupling of carboxylic acids of any one of Embodiments 1-18, wherein the solvent contains a carbonyl moiety.

Embodiment 20. The method of decarboxylative coupling of carboxylic acids of Embodiment 19, wherein the carbonyl moiety is a (C₃-C₁₅)ketone.

Embodiment 21. The method of decarboxylative coupling of carboxylic acids of Embodiment 20, wherein the (C₃-C₁₅)ketone is acetone.

Embodiment 22. The method of decarboxylative coupling of carboxylic acids of any one of Embodiments 1-21, wherein the alternating frequency is approximately 10 Hz.

Embodiment 23. The method of decarboxylative coupling of carboxylic acids of any one of Embodiments 1-22, wherein the current is set at 1-1000 mA/mmol.

Embodiment 24. The method of decarboxylative coupling of carboxylic acids of Embodiment 23, wherein the current is set at approximately 60 mA/mmol.

Embodiment 25. The method of decarboxylative coupling of carboxylic acids of any one of Embodiments 1-24, wherein the amount of charge is set at 1-20 F/mol.

Embodiment 26. The method of decarboxylative coupling of carboxylic acids of Embodiment 25, wherein the amount of charge is set at approximately 8-10 F/mol.

Embodiment 27. The method of decarboxylative coupling of carboxylic acids of any one of Embodiments 1-26, wherein the electrodes are RVC electrodes.

Embodiment 28. The method of decarboxylative coupling of carboxylic acids of any one of Embodiments 1-27, wherein the carboxylic acid substrate comprises one carboxylic acid or two carboxylic acids.

Embodiment 29. The method of decarboxylative coupling of carboxylic acids of Embodiment 28, wherein the carboxylic acid substrate comprises one carboxylic acid.

Embodiment 30. The method of decarboxylative coupling of carboxylic acids of any one of Embodiments 1-29, wherein the rAP-Kolbe electrolysis occurs according to the following reaction scheme:

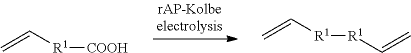

wherein R¹ is (C₁-C₂₀)alkyl or cycloalkyl, optionally substituted with aryl, heteroaryl, O, N, or halo.

Embodiment 31. The method of decarboxylative coupling of carboxylic acids of any one of Embodiments 1-29, wherein the rAP-Kolbe electrolysis occurs according to the following reaction scheme:

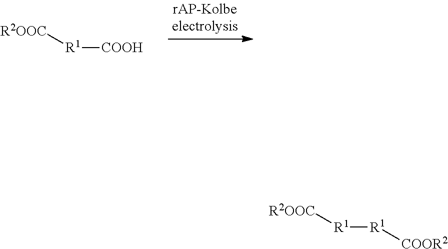

wherein R¹ is (C₁-C₂₀)alkyl or cycloalkyl, optionally substituted with aryl, heteroaryl, O, N, or halo; and
R² is (C₁-C₆)alkyl or cycloalkyl.

Embodiment 32. The method of decarboxylative coupling of carboxylic acids of any one of Embodiments 1-29, wherein the rAP-Kolbe electrolysis occurs according to the following reaction scheme:

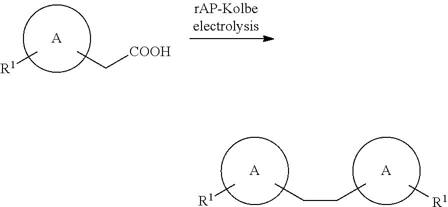

wherein A is (C₃-C₂₀)cycloalkyl; and
R¹ is (C₁-C₂₀)alkyl or (C₃-C₂₀)cycloalkyl, optionally substituted with aryl, heteroaryl, O, N, or halo.

Embodiment 33. The method of decarboxylative coupling of carboxylic acids of any one of Embodiments 1-29, wherein the rAP-Kolbe electrolysis occurs according to the following reaction scheme:

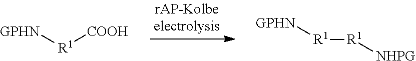

wherein R¹ is (C₁-C₂₀)alkyl, cycloalkyl, or alkyl, optionally substituted with aryl, heteroaryl, O, N, or halo; and
PG is a protecting group including, but not limited to Boc and Cbz.

Embodiment 34. The method of decarboxylative coupling of carboxylic acids of any one of Embodiments 1-29, wherein the rAP-Kolbe electrolysis occurs according to the following reaction scheme:

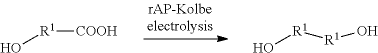

wherein R¹ is (C₁-C₂₀)alkyl, cycloalkyl, or alkyl, optionally substituted with aryl, heteroaryl, O, N, or halo.

Embodiment 35. The method of decarboxylative coupling of carboxylic acids of any one of Embodiments 1-29, wherein the rAP-Kolbe electrolysis occurs according to the following reaction scheme:

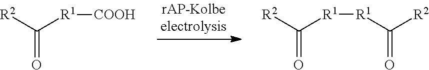

R¹ is (C₁-C₂₀)alkyl or cycloalkyl, optionally substituted with aryl, heteroaryl, O, N, or halo; and
R² is (C₁-C₆)alkyl or cycloalkyl.

Embodiment 36. The method of decarboxylative coupling of carboxylic acids of any one of Embodiments 1-29, wherein the rAP-Kolbe electrolysis occurs according to the following reaction scheme:

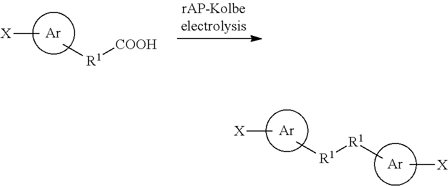

wherein X is halo or OR²;
Ar is aryl, including, but not limited to, Ph and naphthyl;
R¹ is (C₁-C₂₀)alkyl or cycloalkyl, optionally substituted with aryl, heteroaryl, O, N, or halo; and
R² is (C₁-C₆)alkyl or cycloalkyl.

Embodiment 37. The method of decarboxylative coupling of carboxylic acids of any one of Embodiments 1-29, wherein the rAP-Kolbe electrolysis occurs according to the following reaction scheme:

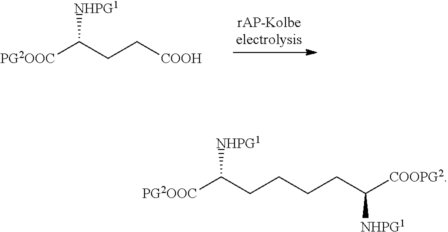

wherein PG¹ is an amino protecting group, including, but not limited to, Boc and Cbz; and PG² is a carboxylic acid protecting group, including, but not limited to, tBu, Me, and Bn.

Embodiment 38. The method of decarboxylative coupling of carboxylic acids of any one of Embodiments 1-29, wherein the rAP-Kolbe electrolysis occurs according to the following reaction scheme:

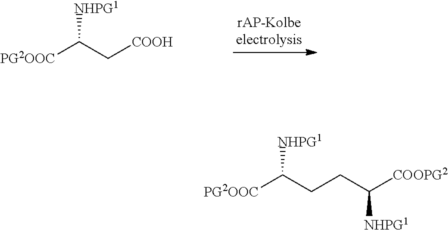

wherein PG¹ is an amino protecting group, including, but not limited to, Boc and Cbz; and
PG² is a carboxylic acid protecting group, including, but not limited to, tBu, Me, and Bn.

Embodiment 39. The method of decarboxylative coupling of carboxylic acids of any one of Embodiments 1-29, wherein the rAP-Kolbe electrolysis occurs according to the following reaction scheme:

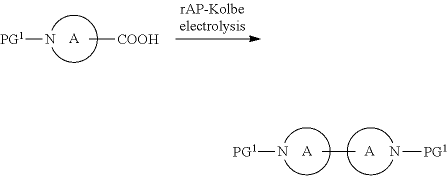

wherein A is (C₃-C₁₀)heterocycle;
PG¹ is an amino protecting group, including, but not limited to, Boc and Cbz.

Embodiment 40. The method of decarboxylative coupling of carboxylic acids of any one of Embodiments 1-39, wherein the carboxylic acid substrate concentration is approximately 1.5 mmol.

Embodiment 41. The method of decarboxylative coupling of carboxylic acids of any one of Embodiments 1-40, wherein the carboxylic acid substrate is treated with approximately 10 mol % NH₄OH.

Embodiment 42. The method of decarboxylative coupling of carboxylic acids of any one of Embodiments 1-40, wherein the solvent is acetone.

Embodiment 43. The method of decarboxylative coupling of carboxylic acids of any one of Embodiments 40-42, wherein the current is set at approximately 60 mA.

Embodiment 44. The method of decarboxylative coupling of carboxylic acids of any one of Embodiments 40-43, wherein the alternating frequency is set at approximately 10 Hz (50 ms).

Embodiment 45. The method of decarboxylative coupling of carboxylic acids of any one of Embodiments 40-44, wherein the amount of charge is set at approximately 8 F/mol.

Embodiment 46. The method of decarboxylative coupling of carboxylic acids of any one of Embodiments 40-45, wherein the electrodes are RVC electrodes.

Embodiment 47. The method of decarboxylative coupling of carboxylic acids of any one of Embodiments 1-39, wherein the carboxylic acid substrate concentration is approximately 200 mmol.

Embodiment 48. The method of decarboxylative coupling of carboxylic acids of Embodiment 47, wherein the carboxylic acid substrate is treated with approximately 10 mol % NH₄OH.

Embodiment 49. The method of decarboxylative coupling of carboxylic acids of either Embodiment 46 or Embodiment 47, wherein the solvent is acetone.

Embodiment 50. The method of decarboxylative coupling of carboxylic acids of any one of Embodiments 47-49, wherein the current is set at approximately 60 mA.

Embodiment 51. The method of decarboxylative coupling of carboxylic acids of any one of Embodiments 47-50, wherein the alternating frequency is set at approximately 3.3 Hz (150 ms).

Embodiment 52. The method of decarboxylative coupling of carboxylic acids of any one of Embodiments 47-51, wherein the amount of charge is set at approximately 8 F/mol.

Embodiment 53. The method of decarboxylative coupling of carboxylic acids of any one of Embodiments 47-52, wherein the electrodes are RVC electrodes.

Embodiment 54. The method of decarboxylative coupling of carboxylic acids of any one of Embodiments 1-28, wherein the carboxylic acid substrate comprises two carboxylic acids.

Embodiment 55. The method of decarboxylative coupling of carboxylic acids of Embodiment 54, wherein one or both of the two different carboxylic acids is an amino acid or derivative thereof.

Embodiment 56. The method of decarboxylative coupling of carboxylic acids of Embodiment 54, wherein the rAP-Kolbe electrolysis occurs according to the following reaction scheme:

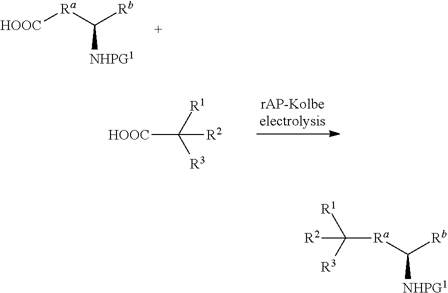

wherein each of R^{a} and R^{b} is independently selected from (C₁-C₂₀)alkyl or cycloalkyl, optionally substituted with aryl, heteroaryl, O, N, or halo; and
PG¹ is an amino protecting group, including, but not limited to, Boc and Cbz.

Embodiment 57. The method of decarboxylative coupling of carboxylic acids of Embodiment 54, wherein the rAP-Kolbe electrolysis occurs according to the following reaction scheme:

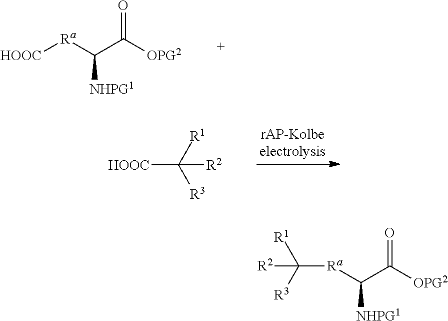

wherein R^{a} is (C₁-C₂₀)alkyl or cycloalkyl, optionally substituted with aryl, heteroaryl, O, N, or halo;
PG¹ is an amino protecting group, including, but not limited to, Boc and Cbz; and
PG² is a carboxylic acid protecting group, including, but not limited to, tBu, Me, and Bn.

Embodiment 58. The method of decarboxylative coupling of carboxylic acids of Embodiment 54, wherein the rAP-Kolbe electrolysis occurs according to the following reaction scheme:

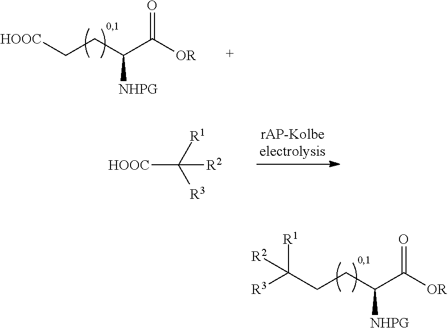

wherein PG is an amino protecting group, including, but not limited to, Boc and Cbz;
R is (C₁-C₆)alkyl or (C₁-C₆)alkylaryl; and
each of R¹, R², and R³ is independently selected from H, (C₁-C₁₂)alkyl, (C₁-C₁₂)haloalkyl, (C₂-C₁₂)alkenyl, (C₁-C₆)alkyl ester, and (C₁-C₆)alkyl-NHBoc.

Embodiment 59. The method of decarboxylative coupling of carboxylic acids of any one of Embodiments 54-58, wherein the carboxylic acid substrate concentration is approximately 1.5 mmol.

Embodiment 60. The method of decarboxylative coupling of carboxylic acids of any one of Embodiments 54-59, wherein the carboxylic acid substrate is treated with approximately 10 mol % NMe₄OH.

Embodiment 61. The method of decarboxylative coupling of carboxylic acids of any one of Embodiments 54-60, wherein the carboxylic acid substrate is treated with approximately 2 equivalents of NMe₄BF₄.

Embodiment 62. The method of decarboxylative coupling of carboxylic acids of any one of Embodiments 54-61, wherein the solvent is acetone.

Embodiment 63. The method of decarboxylative coupling of carboxylic acids of any one of Embodiments 54-62, wherein the current is set at approximately 60 mA.

Embodiment 64. The method of decarboxylative coupling of carboxylic acids of any one of Embodiments 54-63, wherein the alternating frequency is set at approximately 10 Hz (50 ms).

Embodiment 65. The method of decarboxylative coupling of carboxylic acids of any one of Embodiments 54-64, wherein the amount of charge is set at approximately 10 F/mol.

Embodiment 66. The method of decarboxylative coupling of carboxylic acids of any one of Embodiments 54-65, wherein the electrodes are RVC electrodes.

Embodiment 67. A method of decarboxylative olefination of carboxylic acids, wherein the carboxylic acid substrate is subjected to rAP electrolysis.

Embodiment 68. The method of decarboxylative olefination of carboxylic acids of Embodiment 67, wherein the rAP electrolysis occurs according to either reaction scheme (i) or reaction scheme (ii):

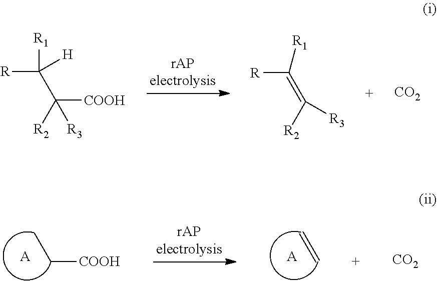

wherein R¹, R² and R³ are optionally H or substituted (C₁-C₂₀)alkyl, (C₂-C₂₀)alkenyl, (C₁-C₂₀)hydroxyalkyl, and N-protected (C₁-C₂₀)aminoalkyl; and
A is optionally substituted, saturated or partially unsaturated, (C₃-C₈)cycloalkyl or (C₃-C₈)heterocycloalkyl.

Embodiment 69. The method of decarboxylative olefination of carboxylic acids of either Embodiment 67 or Embodiment 68, wherein the concentration of the carboxylic acid substrate is approximately 1.5 mmol.

Embodiment 70. The method of decarboxylative olefination of carboxylic acids of any one of Embodiments 67-69, wherein the carboxylic acid substrate is treated with approximately 30 mol % tetramethyl ammonium hydroxide.

Embodiment 71. The method of decarboxylative olefination of carboxylic acids of any one of Embodiments 67-70, wherein the carboxylic acid substrate is treated with approximately 20 mol % pivOH.

Embodiment 72. The method of decarboxylative olefination of carboxylic acids of any one of Embodiments 67-71, wherein the solvent is acetone.

Embodiment 73. The method of decarboxylative olefination of carboxylic acids of any one of Embodiments 67-72, wherein the current is set at approximately 100 mA.

Embodiment 74. The method of decarboxylative olefination of carboxylic acids of any one of Embodiments 67-73, wherein the alternating frequency is set at approximately 5 s.

Embodiment 75. The method of decarboxylative olefination of carboxylic acids of any one of Embodiments 67-74, wherein the amount of charge is set at 5 F/mol.

Embodiment 76. The method of decarboxylative olefination of carboxylic acids of any one of Embodiments 67-75, wherein the electrodes are Graphite electrodes.

Embodiment 77. The method of decarboxylative olefination of carboxylic acids of any one of Embodiments 67-76, wherein the rAP electrolysis occurs according to the following reaction scheme:

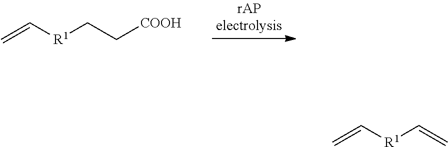

wherein R¹ is (C₁-C₂₀)alkyl or cycloalkyl, optionally substituted with aryl, heteroaryl, O, N, or halo.

Embodiment 78. The method of decarboxylative olefination of carboxylic acids of any one of Embodiments 67-77, wherein the rAP electrolysis occurs according to the following reaction scheme:

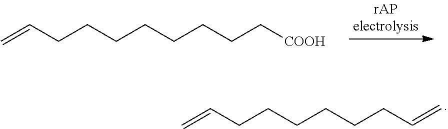

Embodiment 79. The method of decarboxylative olefination of carboxylic acids of any one of Embodiments 67-76, wherein the rAP-Kolbe electrolysis occurs according to the following reaction scheme:

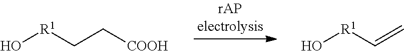

wherein R¹ is (C₁-C₂₀)alkyl or cycloalkyl, optionally substituted with aryl, heteroaryl, O, N, or halo.

Embodiment 80. The method of decarboxylative olefination of carboxylic acids of any one of Embodiments 67-76 and 79, wherein the rAP-Kolbe electrolysis occurs according to the following reaction scheme:

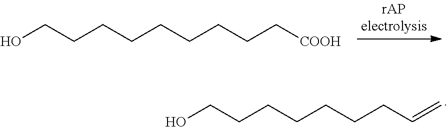

Embodiment 81. The method of decarboxylative olefination of carboxylic acids of any one of Embodiments 67-76, wherein the rAP-Kolbe electrolysis occurs according to the following reaction scheme:

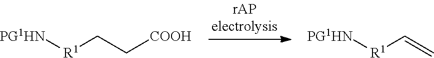

R¹ is (C₁-C₂₀)alkyl or cycloalkyl, optionally substituted with aryl, heteroaryl, O, N, or halo; and
PG¹ is an amino protecting group, including, but not limited to, Boc and Cbz.

Embodiment 82. The method of decarboxylative olefination of carboxylic acids of any one of Embodiments 67-76 and 81, wherein the rAP-Kolbe electrolysis occurs according to the following reaction scheme:

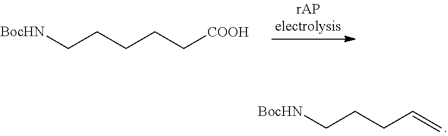 [SMILES: CC(C)(C)OC(=O)NCCCCCC(=O)O ; C=CCCCNC(=O)OC(C)(C)C]

Embodiment 83. The method of decarboxylative olefination of carboxylic acids of any one of Embodiments 67-76, wherein the rAP-Kolbe electrolysis occurs according to the following reaction scheme:

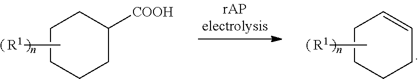

each R¹ is independently (C₁-C₂₀)alkyl or cycloalkyl, optionally substituted with aryl, heteroaryl, O, N, or halo;
or two R¹ together form (C₄-C₁₂)cycloalkyl, optionally substituted with (C₁-C₂₀)alkyl, cycloalkyl, aryl, heteroaryl, O, N, or halo; and
   n is 0, 1, or 2.

Embodiment 84. A composition obtained by the decarboxylative coupling of carboxylic acids of any one of Embodiments 1-53, wherein the composition is formed according to the following reaction:

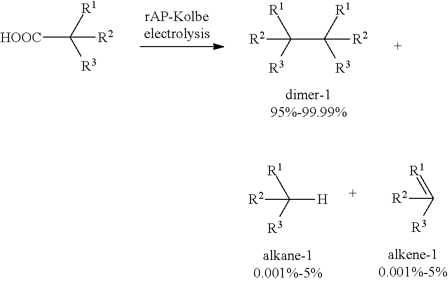

wherein R¹, R², and R³ are each independently C₁-C₅₀ alkyl, optionally substituted with heteroatoms.

Embodiment 85. A composition obtained by the decarboxylative coupling of carboxylic acids of any one of Embodiments 54-66, wherein the composition is formed according to the following reaction:

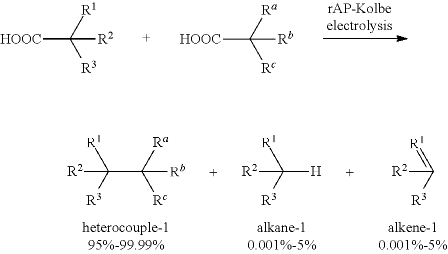

wherein R¹, R², R³, R^{a}, R^{b}, and R^{c} are each independently C₁-C₅₀ alkyl, optionally substituted with heteroatoms.

Embodiment 86. Any 1) method of decarboxylative coupling of a carboxylic acid substrate, wherein the carboxylic acid substrate comprises either one carboxylic acid or two carboxylic acids and subjected to rAP-Kolbe electrolysis; 2) method of decarboxylative olefination of a carboxylic acid substrate, wherein the carboxylic acid substrate is subjected to rAP electrolysis; 3) composition obtained by decarboxylative coupling of carboxylic acids; or 4) any method of preparing chemicals including monomers, polymers, additives, adhesives, solvents, pharmaceuticals, amino acids and derivatives thereof, agricultural chemicals, fragrances, fuels, lubricants, or electronic materials according to the above methods, as disclosed herein.

### Definitions

### Kolbe Electrolysis:

The term “Kolbe electrolysis,” as used herein, refers to the electrolytic oxidative decarboxylation of a solvated aliphatic carboxylic acid to generate a transient alkyl radical, which combines with another transient alkyl radical to form a Csp³-Csp³ bond via radical-radical coupling. Such a transformation is tremendously useful due to the wide availability of carboxylic acids and necessity of no exogenous costly additives to facilitate decarboxylation. Most importantly, the Kolbe electrolysis holds promise to drastically shorten synthesis by avoiding excessive protecting/redox manipulations typically associated with conventional Csp³-Csp³ bond formation tactics such as Grignard addition or Wittig-type olefinations followed by hydrogenation. Despite these attractive features, this reaction has had little success in practical synthesis due to the poor functional group compatibility resulting from the extremely high overpotentials employed and the requirement of expensive Pt electrodes. The low chemoselectivity can be directly traced to the harsh electrolysis conditions employed, which severely limit the menu of compatible functional groups (only esters and ethers). Even compatibility with a seemingly inert terminal olefin is problematic as discussed below. In addition to this limited functional group compatibility, a Pt anode is considered essential for performing the reaction. This is commonly explained by the unique property of Pt surfaces to adsorb carboxylates to facilitate its oxidation over solvent oxidation. Although alternative inexpensive anode materials are a subject of active research, no practical solution has yet been found.

Rapid Alternating Polarity (rAP):

The term “rapid Alternating Polarity (rAP)” as used herein refers to a new electrolysis mode for synthetic organic electrochemistry. “Alternating Polarity” means that the electric current periodically changes its direction. “rapid” means the frequency (Hz) of alternating polarity is high. The appropriate range of the frequency (Hz) is different from each reaction and described later.

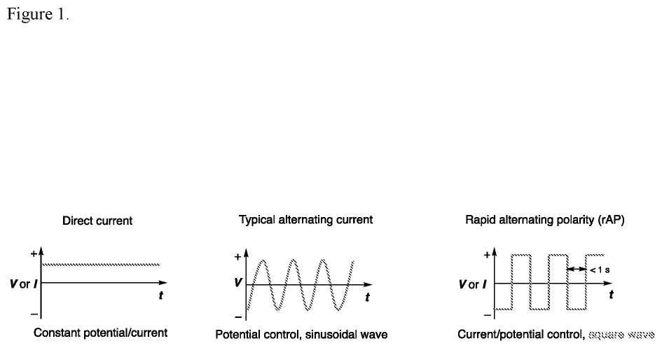

Traditionally, a direct current was applied to electrochemical synthesis of organic compounds, where electricity is applied by holding either current (I) or potential (V) at a constant value (FIG. 1, left). Although much less common, alternating current instead of direct current can be applied to electrochemical synthesis as well. Alternating current refers to electric current that periodically changes its direction. For example, the potential change of alternating current follows a sine-wave pattern (FIG. 1, middle) as well as square waveform (FIG. 1, right). Square waveform can be easily accessed by simply alternating the electrode polarity; thus, we refer this waveform as rapid alternating polarity (rAP) to distinguish it from other type of AC waveform. Although, application of AC waveforms into organic synthesis has received little attention, we recently discovered that AC waveforms, particularly rAP, could profoundly alter the reaction outcome under certain circumstances such as reduction of carbonyl groups and arenes, exhibiting unprecedented levels of chemoselectivity that is absent when DC is used under otherwise identical reaction conditions. There have been publications using rAP-based methods of electrolysis for reductive processes (see references 3 and 4), as opposed to the oxidative rAP-Kolbe electrolysis as disclosed herein.

### Rap-Kolbe Electrolysis:

The term “rAP-Kolbe electrolysis” as used herein refers to Kolbe electrolysis that is carried out by rapid Alternating Polarity (rAP).

### Rap Electrolysis:

The term “rAP electrolysis” as used herein refers to electrolysis that is carried out by rapid Alternating Polarity (rAP) in a solvent.

### Alternating Frequencies:

The term “alternating frequency,” as used herein, in reference to the rAP electrolysis reactions is preferably 1 MHz-0.0001 Hz. As an upper limit, the more preferable alternating frequencies are 1 MHz, 1 kHz, and 100 Hz, with the more preferable alternating frequency being 50 Hz. As a lower limit, 0.0001 Hz, 0.001 Hz, and 0.01 Hz may be used, with the more preferable alternating frequencies being 0.1 Hz or 1 Hz. The frequency correlates with reaction efficiency. If the alternating frequency is too high, the reaction conversion decreases due to lower current efficiency. If the alternating frequency is too low, undesired side reactions, such as unselective oxidation or electrode passivation, may occur, as such low-frequency rAP is similar to DC electrolysis.

### Current Waveforms:

As used herein, the term “current waveform” is preferably a square waveform for the most efficient electrolytic conditions. Other waveforms, such as a sinusoidal waveform, may exhibit similar effects in rAP electrolysis reactions as disclosed herein, however with lower current efficiencies.

### Electrodes:

As used herein, the rAP electrolysis electrode materials include, but are not limited to, metallic materials such as Pt, Pd, Ni, Rh, Ti, Pb, conductive metal oxides, or carbon-based materials including surface modifications. Carbon-based electrode materials are preferable in terms of reaction selectivity, reaction rate, surface area, chemical durability, and price. Carbon-based materials for electrode materials, as used herein, includes, but is not limited to, graphite, carbon-fibers, carbon-felt, GC (glassy carbon), RVC (Reticulated Vitreous Carbon), diamond with various dopings to increase conductivity, and carbon-layer deposited electrodes. Among the carbon-based material, graphite, GC (grassy carbon), RVC (Reticulated Vitreous Carbon) are preferable in terms of reaction efficiency. RVC is especially preferable due to its high surface area.

### Electrolysis Reactors:

The reactor size is not limited and may be selected according to the reaction scale. As for types of reactor design, all common choices of divided or undivided, batch or flow reactors are acceptable. In particular, a simple undivided cell equipped with two RVC electrodes is most preferable for a convenient set-up.

### Bases:

The reactions using rAP electrolysis as described herein proceed most effectively in the presence of a base in the reaction solution. As used herein, the term “base” includes metal hydroxides (such as NaOH, KOH, CsOH), metal carbonates, metal acetates, ammonium hydroxides (such as NR₄OH, NH₄OH, wherein R is (C₁-C₆)alkyl), and amine containing compounds). Among bases, NR₄OH, NH₄OH, NaOH, and KOH, wherein R is (C₁-C₆)alkyl, are preferable with respect to the base strength and affordability. NR₄OH is especially preferable in terms of solubility in organic solvents and stability under rAP electrolysis. Additionally, the base may comprise a single base or a mixture of bases. The preferable amount of base is approximately 0.01 mol % to approximately 200 mol % to the overall amount of the carboxylic acid substrate(s). More preferably, the amount of base is approximately 10-30 mol %. Either too little or too much base negatively affects the rAP electrolysis reaction efficiency.

### Solvents:

As disclosed herein, the rAP electrolysis reactions occur in the presence of a solvent. The preferable amount of solvent is around 0.1-50 mL of solvent per 1 mmol of carboxylic acid. If the concentration is too high, the substrate might not completely dissolve. If the concentration is too low, radical-radical coupling may not occur efficiently. The term “solvent,” as used herein to describe the rAP electrolysis reactions, includes, but is not limited to, THF, DMF, DMSO, water, methanol, acetone, acetonitrile, dicloromethane, nitromethane, NMP, and DMAc. The relative dielectric constant of the solvent is preferably >5. Solvents containing a carbonyl moiety are preferable with respect to the solubility of the substrate and base, as well as the prevention of side reactions. With respect to the number of carbons of the carbonyl moiety, (C₃-C₁₅)ketones are preferable in terms of the stability under electrolysis. Acetone is most preferable as a solvent. Additionally, the solvent may be a single solvent or a mixture of two or more solvents.

### Supporting Electrolytes:

Supporting electrolytes may be used to facilitate the rAP electrolysis reactions more effectively or to decrease the resistance of the reaction mixture. As supporting electrolytes, metal salts (including Li, Na, K, Cs, Mg, and Ca salts comprising halogen anions are used), ammonium salts (including NR₄OH, NR₄BF₄, NR₄PF₆) and phosphonium salts are included. Among the supporting electrolytes, ammonium salts are preferable, in terms of the solubility, and NR₄BF₄ is more preferable in terms of the stability under electrolysis and the prevention of side reactions. The concentration of the supporting electrolyte is preferably 0.01-10 M. Bases, or other additives may also serve as support electrolytes by forming ion pairs with the carboxylic acid during the rAP electrolysis reaction.

### Other Additives:

Other additives may be used to facilitate the rAP electrolysis reactions of the disclosure more effectively or to decrease the resistance of the reaction mixture. Exemplary additives include acids (to control pH), passivation preventive agents, and decarboxylation activators. Among acid additives, pivalic acid is preferable because of low reactivity and good solubility. Passivation preventive agents include nitrogen containing compounds (such as amine derivatives, urea derivatives, and pyridine derivatives) and phosphorus containing compounds (such as phosphoric acid esters and phosphoric acid amides) which are preferable because of high durability under rAP electrolysis conditions. Decarboxylation activators include nitrogen containing compounds (such as amine derivatives, urea derivatives, and pyridine derivatives) and phosphorus containing compounds (such as phosphoric acid esters and phosphoric acid amides) which are preferable because of high durability under rAP electrolysis conditions.

### Current:

As used herein the term “current” is not defined as limited to a specific value per substrate amount or electrode type. However, a preferable current range as per mol is between 1-1000 mA/mmol. A preferable current range per surface area is 0.001-1000 mA/cm². For example, on 1.5 mmol scale, approximately 20 mA/cm² is preferable. If the current is too low, the reaction is either too slow or does not occur. If the current is too high, side reactions or passivation of electrodes may occur.

### Amount of Charge:

As intended herein, the term “amount of charge” is not limited to a specific value if sufficient yield of the desired product is obtained. Preferable ranges include 1-20 F/mol, and most preferably 8-10 F/mol. If the amount of charge is too low, the reaction may be incomplete. If the amount of charge is too high, side reactions and product decomposition may occur.

### Carboxylic Acid Substrates:

The term “carboxylic acid substrate,” as used herein, refers to the carboxylic acid that is the starting material for the rAP-Kolbe electrolysis reaction for decarboxylative coupling of carboxylic acids, wherein the rAP-Kolbe electrolysis occurs according to either reaction scheme (i) or reaction scheme (ii):

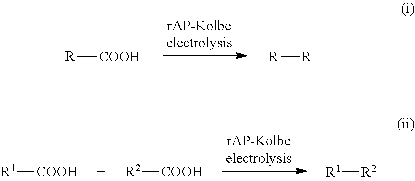

wherein each of R, R¹, and R² is independently selected from H, saturated or unsaturated acyclic or cyclic aliphatic groups, each optionally including one or more heteroatoms and each optionally substituted with alkyl, alkenyl, ester, amino, halo, hydroxy, keto, formyl, aryl, heteroaryl, cycloalkyl or heterocyclic moieties, and aromatic or heteroaromatic ring systems, each optionally substituted with alkyl, alkenyl, ester, amino, halo, hydroxy, keto, formyl, aryl, heteroaryl, cycloalkyl or heterocyclic moieties.

Carboxylic acids containing hetero atoms or unsaturated bonds are useful in producing otherwise difficult to synthesize products such as certain chemicals or Pharmaceuticals. Also, under traditional Kolbe reaction conditions, it is difficult to obtain such hetero atom or unsaturated bond containing products because of side reactions or the passivation of the electrodes. It is preferable that the carboxylic acid substrate contains 2-100 carbon atoms, and even more preferably, 2-50 atoms. The number of substituents on the carboxylic acid is not limited in order for the rAP-Kolbe electrolysis reactions to proceed efficiently.

rAP-Kolbe electrolysis reactions using natural products, and derivatives thereof, as the carboxylic acid substrate, are also useful, especially those containing chiral carbon, for example amino acid, tartaric acid, fatty acids, and the like, and derivatives thereof. They are useful for producing high value materials such as chemicals, polymers, pharmaceuticals, and fuels, for example, from sustainable resources. Carboxylic acids may comprise a single carboxylic acid or two different carboxylic acids in a substrate. If single carboxylic acid is used, a dimerization product is obtained. If two carboxylic acids are used, heterocoupling products are obtained (see below).

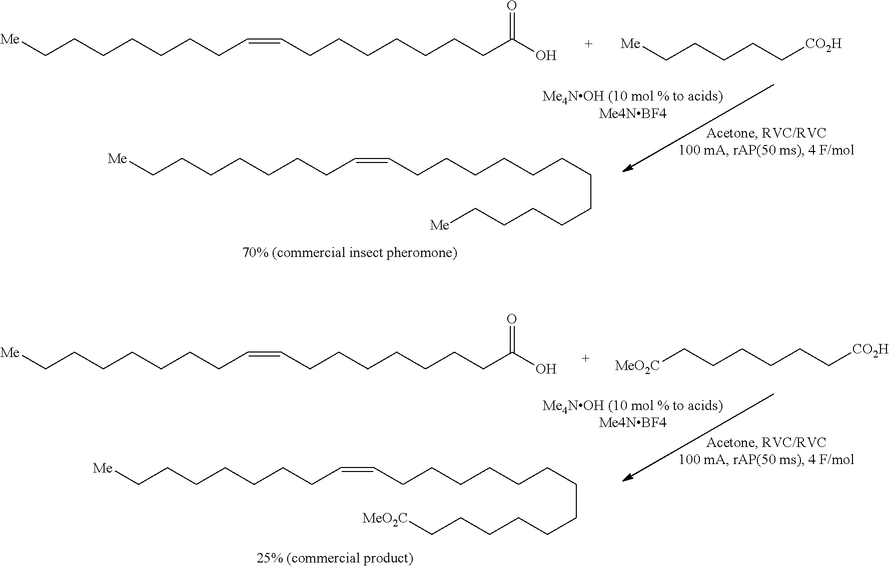

### Compositions of the Product:

As the rAP-Kolbe electrolysis occurs, some byproducts may be produced in addition to the desired dimer product. To increase the purity of the desired product, standard purification methods may be carried out if needed such as extraction, crystallization, column chromatography, and distillation. Notably, the crude compositions obtained by the reaction may be beneficial for subsequent reactions or other purposes. For example, the resulting reaction compositions comprising the desired homocoupling dimer components (dimer-1, obtained at 95.0%-99.99%, wherein % refers to weight %), along with alkyl byproducts (alkane-1, obtained as a byproduct at 0.001-5%), as well as alkenyl byproducts (alkene-1, obtained as a byproduct at 0.001-5%), is a satisfactory starting material composition for subsequent reactions, including polymerizations. The compositions have a higher solubility in organic solvents than the pure desired dimer products. Thus, use of the crude compositions allows subsequent reactions, including polymerizations, to proceed more efficiently. Also, the compositions may preferably be used from an economic perspective, because the dimer-, alkane-1, and alkene-1, all have similar physical properties which makes it difficult to separate them completely. The weight % of the different components of the composition are analyzed by NMR, GC, LC, MS, or FTIR.

The application provides a composition obtained by the decarboxylative coupling of carboxylic acids of any one of Embodiments 1-53, wherein the composition is formed according to the following reaction:

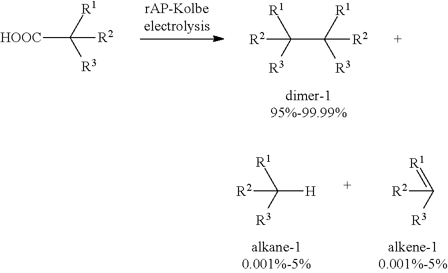

wherein R¹, R², and R³ are each independently C₁-C₅₀ alkyl, optionally substituted with heteroatoms.

The application further provides a composition obtained by the decarboxylative coupling of carboxylic acids of any one of Embodiments 54-66, wherein the composition is formed according to the following reaction:

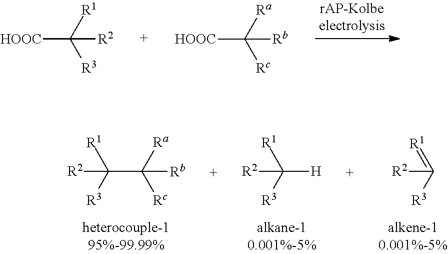

wherein R¹, R², R³, R^{a}, R^{b}, and R^{c} are each independently C₁-C₅₀ alkyl, optionally substituted with heteroatoms.

### Applications of the Product:

The products of the rAP-Kolbe electrolysis reactions may be useful in various applications. The instant disclosure is not limited to the specific reactions described below as representative examples. Subsequent reactions with the rAP-Kolbe electrolysis products may be used to form useful chemicals, including monomers, polymers, agricultural chemicals, additives, adhesives, solvents, pharmaceuticals, amino acids and derivatives thereof, fragrances, fuels, lubricants, electronic materials, as well as other useful applications which are further included.

Conversion of rAP-Kolbe Electrolysis Reaction Products:

The application provides the method of decarboxylative coupling of carboxylic acids of any one of Embodiments 1-29, wherein the rAP-Kolbe electrolysis occurs according to the following general reaction scheme:

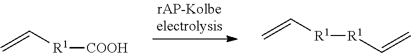

wherein R¹ is (C₁-C₂₀)alkyl or cycloalkyl, optionally substituted with aryl, heteroaryl, O, N, or halo.

Further conversions of the rAP-Kolbe electrolysis products obtained from the above reaction scheme, as a general example, include the product being further converted into other products, including, but not limited to, the substitution of the rAP-Kolbe electrolysis product with substituents such as alkenyl, ester, amino, halo, hydroxy, keto, formyl, aryl, heteroaryl, cycloalkyl or heterocyclic moieties, as well as aromatic or heteroaromatic ring systems. The rAP-Kolbe electrolysis reaction products can be converted by further substitution via various reactions including polymerizations (such as oxidation or reduction or addition or cycloaddition or rearrangement). For example, rAP-Kolbe electrolysis reaction product alkenes may readily be converted via further substitution reactions, such as those incorporating epoxy, hydroxyl, boryl, silyl, bromo, amino, and carboxyl moieties by simple processes that are provided for as well-known in the art and in good yields.

### Decarboxylative Olefination:

Further provided herein is a method of decarboxylative olefination of carboxylic acids, wherein the carboxylic acid substrate is subjected to rAP electrolysis.

The application further provides the above method of decarboxylative olefination of carboxylic acids, wherein the rAP electrolysis occurs according to either reaction scheme (i) or reaction scheme (ii):

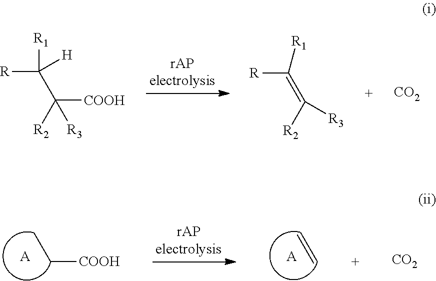

wherein R¹, R² and R³ are optionally H or substituted (C₁-C₂₀)alkyl, (C₂-C₂₀)alkenyl, (C₁-C₂₀)hydroxyalkyl, and N-protected (C₁-C₂₀)aminoalkyl; and
A is optionally substituted, saturated or partially unsaturated, (C₃-C₈)cycloalkyl or (C₃-C₈)heterocycloalkyl.

### Reaction Conditions for Decarboxylative Olefination:

Reaction conditions for decarboxylative olefination is not limited in scope and the product is obtained in satisfactory yields. rAP electrolysis is preferable described herein. As for preferred electrodes, included are carbon-based materials, as preferable in terms of reaction selectivity, reaction rate, surface area, chemical durability and price. Carbon-based materials include, but are not limited to, Graphite, carbon-fibers, carbon-felt, GC (glassy carbon), RVC (Reticulated Vitreous Carbon), and diamond electrodes with various dopants to increase conductivity. Among the carbon-based materials, Graphite is the most preferable in terms of reaction efficiency.

### General Definitions

The phrase “a” or “an” entity as used herein refers to one or more of that entity; for example, a compound refers to one or more compounds or at least one compound. As such, the terms “a” (or “an”), “one or more”, and “at least one” can be used interchangeably herein.

The phrase “as defined herein above” refers to the broadest definition for each group as provided in the Summary of the Invention, the Detailed Description of the Invention, the Experimentals, or the broadest claim. In all other embodiments provided below, substituents which can be present in each embodiment and which are not explicitly defined retain the broadest definition provided in the Summary of the Invention.

As used in this specification, whether in a transitional phrase or in the body of the claim, the terms “comprise(s)” and “comprising” are to be interpreted as having an open-ended meaning. That is, the terms are to be interpreted synonymously with the phrases “having at least” or “including at least”. When used in the context of a process, the term “comprising” means that the process includes at least the recited steps, but may include additional steps. When used in the context of a compound or composition, the term “comprising” means that the compound or composition includes at least the recited features or components, but may also include additional features or components.

As used herein, unless specifically indicated otherwise, the word “or” is used in the “inclusive” sense of “and/or” and not the “exclusive” sense of “either/or”.

The term “independently” is used herein to indicate that a variable is applied in any one instance without regard to the presence or absence of a variable having that same or a different definition within the same compound. Thus, in a compound in which “R” appears twice and is defined as “independently selected from” means that each instance of that R group is separately identified as one member of the set which follows in the definition of that R group. For example, “each R¹ and R² is independently selected from carbon and nitrogen” means that both R¹ and R² can be carbon, both R¹ and R² can be nitrogen, or R¹ or R² can be carbon and the other nitrogen or vice versa.

When any variable occurs more than one time in any moiety or formula depicting and describing compounds employed or claimed in the present invention, its definition on each occurrence is independent of its definition at every other occurrence. Also, combinations of substituents and/or variables are permissible only if such compounds result in stable compounds.

The symbols “*” at the end of a bond or a line dcruden through a bond or “˜˜˜˜” dcruden through a bond each refer to the point of attachment of a functional group or other chemical moiety to the rest of the molecule of which it is a part.

A bond dcruden into ring system (as opposed to connected at a distinct vertex) indicates that the bond may be attached to any of the suitable ring atoms.

The term “optional” or “optionally” as used herein means that a subsequently described event or circumstance may, but need not, occur, and that the description includes instances where the event or circumstance occurs and instances in which it does not. For example, “optionally substituted” means that the “optionally substituted” moiety may incorporate a hydrogen or a substituent.

The phrase “optional bond” means that the bond may or may not be present, and that the description includes single, double, or triple bonds. If a substituent is designated to be a “bond” or “absent”, the atoms linked to the substituents are then directly connected.

The term “approximately” is used herein to mean about, in the region of, roughly, or around. When the term “approximately” is used in conjunction with a numerical range, it modifies that range by extending the boundaries above and below the numerical values set forth. In general, the term “approximately” is used herein to modify a numerical value above and below the stated value by a variance of 20%.

Certain compounds of the disclosure may exhibit tautomerism. Tautomeric compounds can exist as two or more interconvertable species. Prototropic tautomers result from the migration of a covalently bonded hydrogen atom between two atoms. Tautomers generally exist in equilibrium and attempts to isolate individual tautomers usually produce a mixture whose chemical and physical properties are consistent with a mixture of compounds. The position of the equilibrium is dependent on chemical features within the molecule. For example, in many aliphatic aldehydes and ketones, such as acetaldehyde, the keto form predominates while; in phenols, the enol form predominates. Common prototropic tautomers include keto/enol (—C(═O)—CH—⇄—C(—OH)═CH—), amide/imidic acid (—C(═O)—NH—⇄—C(—OH)═N—) and amidine (—C(═NR)—NH—⇄-C(—NHR)═N—) tautomers. The latter two are particularly common in heteroaryl and heterocyclic rings and the present invention encompasses all tautomeric forms of the compounds.

Technical and scientific terms used herein have the meaning commonly understood by one of skill in the art to which the present invention pertains, unless otherwise defined. Reference is made herein to various methodologies and materials known to those of skill in the art. Standard reference works setting forth the general principles of pharmacology include Goodman and Gilman's The Pharmacological Basis of Therapeutics, 10^{th} Ed., McGcrude Hill Companies Inc., New York (2001). Any suitable materials and/or methods known to those of skill can be utilized in carrying out the present invention. However, preferred materials and methods are described. Materials, reagents and the like to which reference are made in the following description and examples are obtainable from commercial sources, unless otherwise noted.

The definitions described herein may be appended to form chemically-relevant combinations, such as “heteroalkylaryl,” “haloalkylheteroaryl,” “arylalkylheterocyclyl,” “alkylcarbonyl,” “alkoxyalkyl,” and the like. When the term “alkyl” is used as a suffix following another term, as in “phenylalkyl,” or “hydroxyalkyl,” this is intended to refer to an alkyl group, as defined above, being substituted with one to two substituents selected from the other specifically-named group. Thus, for example, “phenylalkyl” refers to an alkyl group having one to two phenyl substituents, and thus includes benzyl, phenylethyl, and biphenyl. An “alkylaminoalkyl” is an alkyl group having one to two alkylamino substituents. “Hydroxyalkyl” includes 2-hydroxyethyl, 2-hydroxypropyl, 1-(hydroxymethyl)-2-methylpropyl, 2-hydroxybutyl, 2,3-dihydroxybutyl, 2-(hydroxymethyl), 3-hydroxypropyl, and so forth. Accordingly, as used herein, the term “hydroxyalkyl” is used to define a subset of heteroalkyl groups defined below. The term -(ar)alkyl refers to either an unsubstituted alkyl or an aralkyl group. The term (hetero)aryl or (het)aryl refers to either an aryl or a heteroaryl group.

The term “acyl” as used herein denotes a group of formula —C(═O)R wherein R is hydrogen or lower alkyl as defined herein. The term or “alkylcarbonyl” as used herein denotes a group of formula C(═O)R wherein R is alkyl as defined herein. The term C₁₋₆ acyl refers to a group —C(═O)R contain 6 carbon atoms. The term “arylcarbonyl” as used herein means a group of formula C(═O)R wherein R is an aryl group; the term “benzoyl” as used herein an “arylcarbonyl” group wherein R is phenyl.

The term “alkyl” as used herein denotes an unbranched or branched chain, saturated, monovalent hydrocarbon residue containing 1 to 20 carbon atoms. The term “lower alkyl” or “C₁-C₆ alkyl” as used herein denotes a straight or branched chain hydrocarbon residue containing 1 to 6 carbon atoms. “C₁₋₂₀ alkyl” as used herein refers to an alkyl composed of 1 to 20 carbons. Examples of alkyl groups include, but are not limited to, lower alkyl groups including methyl, ethyl, propyl, i-propyl, n-butyl, i-butyl, t-butyl or pentyl, isopentyl, neopentyl, hexyl, heptyl, and octyl.

When the term “alkyl” is used as a suffix following another term, as in “phenylalkyl,” or “hydroxyalkyl,” this is intended to refer to an alkyl group, as defined above, being substituted with one to two substituents selected from the other specifically-named group. Thus, for example, “phenylalkyl” denotes the radical R′R″—, wherein R′ is a phenyl radical, and R″ is an alkylene radical as defined herein with the understanding that the attachment point of the phenylalkyl moiety will be on the alkylene radical. Examples of arylalkyl radicals include, but are not limited to, benzyl, phenylethyl, 3-phenylpropyl. The terms “arylalkyl” or “aralkyl” are interpreted similarly except R′ is an aryl radical. The terms “(het)arylalkyl” or “(het)aralkyl” are interpreted similarly except R′ is optionally an aryl or a heteroaryl radical.

When a range of values is listed, it is intended to encompass each value and sub-range within the range. For example, “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

“Alkyl” refers to a radical of a straight-chain or branched saturated hydrocarbon group having from 1 to 20 carbon atoms (“C₁₋₂₀ alkyl”). In some embodiments, an alkyl group has 1 to 15 carbon atoms (“C₁₋₁₅ alkyl”). In some embodiments, an alkyl group has 1 to 14 carbon atoms (“C₁₋₁₄ alkyl”). In some embodiments, an alkyl group has 1 to 13 carbon atoms (“C₁₋₁₃ alkyl”). In some embodiments, an alkyl group has 1 to 12 carbon atoms (“C₁₋₁₂ alkyl”). In some embodiments, an alkyl group has 1 to 11 carbon atoms (“C₁₋₁₁ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₅) and the like.

“Alkenyl” or “olefin” refers to a radical of a straight-chain or branched hydrocarbon group having from 2 to 20 carbon atoms and 1, 2, 3, or 4 carbon-carbon double bonds (“C₂₋₂₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like.

“Alkynyl” refers to a radical of a straight-chain or branched hydrocarbon group having from 2 to 20 carbon atoms and one or more carbon-carbon triple bonds (e.g., 1, 2, 3, or 4 triple bonds) (“C₂₋₂₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like.

The terms “haloalkyl” or “halo-lower alkyl” or “lower haloalkyl” refers to a straight or branched chain hydrocarbon residue containing 1 to 6 carbon atoms wherein one or more carbon atoms are substituted with one or more halogen atoms.

The term “alkylene” or “alkylenyl” as used herein denotes a divalent saturated linear hydrocarbon radical of 1 to 10 carbon atoms (e.g., (CH₂)ₙ) or a branched saturated divalent hydrocarbon radical of 2 to 10 carbon atoms (e.g., —CHMe- or —CH₂CH(i-Pr)CH₂—), unless otherwise indicated. Except in the case of methylene, the open valences of an alkylene group are not attached to the same atom. Examples of alkylene radicals include, but are not limited to, methylene, ethylene, propylene, 2-methyl-propylene, 1,1-dimethyl-ethylene, butylene, 2-ethylbutylene.

The term “alkoxy” as used herein means an —O-alkyl group, wherein alkyl is as defined above such as methoxy, ethoxy, n-propyloxy, i-propyloxy, n-butyloxy, i-butyloxy, t-butyloxy, pentyloxy, hexyloxy, including their isomers. “Lower alkoxy” as used herein denotes an alkoxy group with a “lower alkyl” group as previously defined. “C₁₋₁₀ alkoxy” as used herein refers to an —O-alkyl wherein alkyl is C₁₋₁₀.

The term “hydroxyalkyl” as used herein denotes an alkyl radical as herein defined wherein one to three hydrogen atoms on different carbon atoms is/are replaced by hydroxyl groups.

The term “cycloalkyl” as used herein refers to a saturated carbocyclic ring containing 3 to 8 carbon atoms, i.e. cyclopropyl, cyclobutyl, cyclopentyl, cyclohexyl, cycloheptyl or cyclooctyl. “C₃₋₇ cycloalkyl” as used herein refers to an cycloalkyl composed of 3 to 7 carbons in the carbocyclic ring.

The term carboxy-alkyl as used herein refers to an alkyl moiety wherein one, hydrogen atom has been replaced with a carboxyl with the understanding that the point of attachment of the heteroalkyl radical is through a carbon atom. The term “carboxy” or “carboxyl” refers to a —CO₂H moiety.

The term “heteroaryl” or “heteroaromatic” as used herein means a monocyclic or bicyclic radical of 5 to 12 ring atoms having at least one aromatic ring containing four to eight atoms per ring, incorporating one or more N, O, or S heteroatoms, the remaining ring atoms being carbon, with the understanding that the attachment point of the heteroaryl radical will be on an aromatic ring. As well known to those skilled in the art, heteroaryl rings have less aromatic character than their all-carbon counter parts. Thus, for the purposes of the invention, a heteroaryl group need only have some degree of aromatic character. Examples of heteroaryl moieties include monocyclic aromatic heterocycles having 5 to 6 ring atoms and 1 to 3 heteroatoms include, but is not limited to, pyridinyl, pyrimidinyl, pyrazinyl, pyrrolyl, pyrazolyl, imidazolyl, oxazol, isoxazole, thiazole, isothiazole, triazoline, thiadiazole and oxadiaxoline which can optionally be substituted with one or more, preferably one or two substituents selected from hydroxy, cyano, alkyl, alkoxy, thio, lower haloalkoxy, alkylthio, halo, lower haloalkyl, alkylsulfinyl, alkylsulfonyl, halogen, amino, alkylamino, dialkylamino, aminoalkyl, alkylaminoalkyl, and dialkylaminoalkyl, nitro, alkoxycarbonyl and carbamoyl, alkylcarbamoyl, dialkylcarbamoyl, arylcarbamoyl, alkylcarbonylamino and arylcarbonylamino. Examples of bicyclic moieties include, but are not limited to, quinolinyl, isoquinolinyl, benzofuryl, benzothiophenyl, benzoxazole, benzisoxazole, benzothiazole and benzisothiazole. Bicyclic moieties can be optionally substituted on either ring; however the point of attachment is on a ring containing a heteroatom.

The term “heterocyclyl”, “heterocycloalkyl” or “heterocycle” as used herein denotes a monovalent saturated cyclic radical, consisting of one or more rings, preferably one to two rings, including spirocyclic ring systems, of three to eight atoms per ring, incorporating one or more ring heteroatoms (chosen from N,O or S(O)₀₋₂), and which can optionally be independently substituted with one or more, preferably one or two substituents selected from hydroxy, oxo, cyano, lower alkyl, lower alkoxy, lower haloalkoxy, alkylthio, halo, lower haloalkyl, hydroxyalkyl, nitro, alkoxycarbonyl, amino, alkylamino, alkylsulfonyl, arylsulfonyl, alkylaminosulfonyl, arylaminosulfonyl, alkylsulfonylamino, arylsulfonylamino, alkylaminocarbonyl, arylaminocarbonyl, alkylcarbonylamino, arylcarbonylamino, unless otherwise indicated. Examples of heterocyclic radicals include, but are not limited to, azetidinyl, pyrrolidinyl, hexahydroazepinyl, oxetanyl, tetrahydrofuranyl, tetrahydrothiophenyl, oxazolidinyl, thiazolidinyl, isoxazolidinyl, morpholinyl, piperazinyl, piperidinyl, tetrahydropyranyl, thiomorpholinyl, quinuclidinyl and imidazolinyl.

“Heterocyclyl” or “heterocyclic” refers to a group or radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include, without limitation, azirdinyl, oxiranyl, and thiiranyl. Exemplary 4-membered heterocyclyl groups containing 1 heteroatom include, without limitation, azetidinyl, oxetanyl and thietanyl. Exemplary 5-membered heterocyclyl groups containing 1 heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include, without limitation, dioxolanyl, oxathiolanyl and dithiolanyl. Exemplary 5-membered heterocyclyl groups containing 3 heteroatoms include, without limitation, triazolinyl, oxadiazolinyl, and thiadiazolinyl. Exemplary 6-membered heterocyclyl groups containing 1 heteroatom include, without limitation, piperidinyl, tetrahydropyranyl, dihydropyridinyl, and thianyl. Exemplary 6-membered heterocyclyl groups containing 2 heteroatoms include, without limitation, piperazinyl, morpholinyl, dithianyl, and dioxanyl. Exemplary 6-membered heterocyclyl groups containing 3 heteroatoms include, without limitation, triazinanyl. Exemplary 7-membered heterocyclyl groups containing 1 heteroatom include, without limitation, azepanyl, oxepanyl and thiepanyl. Exemplary 8-membered heterocyclyl groups containing 1 heteroatom include, without limitation, azocanyl, oxecanyl and thiocanyl. Exemplary bicyclic heterocyclyl groups include, without limitation, indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

“Aryl” refers to a radical of a monocyclic or polycyclic (e.g., bicyclic or tricyclic) 4n+2 aromatic ring system (e.g., having 6, 10, or 14 pi electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl (α-naphthyl) and 2-naphthyl (β-naphthyl)). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system.

“Heteroaryl” refers to a radical of a 5-14 membered monocyclic or polycyclic (e.g., bicyclic, tricyclic) 4n+2 aromatic ring system (e.g., having 6, 10, or 14 pi electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings.

“Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur.

Exemplary 5-membered heteroaryl groups containing 1 heteroatom include, without limitation, pyrrolyl, furanyl and thiophenyl. Exemplary 5-membered heteroaryl groups containing 2 heteroatoms include, without limitation, imidazolyl, pyrazolyl, oxazolyl, isoxazolyl, thiazolyl, and isothiazolyl. Exemplary 5-membered heteroaryl groups containing 3 heteroatoms include, without limitation, triazolyl, oxadiazolyl, and thiadiazolyl. Exemplary 5-membered heteroaryl groups containing 4 heteroatoms include, without limitation, tetrazolyl.

Exemplary 6-membered heteroaryl groups containing 1 heteroatom include, without limitation, pyridinyl. Exemplary 6-membered heteroaryl groups containing 2 heteroatoms include, without limitation, pyridazinyl, pyrimidinyl, and pyrazinyl. Exemplary 6-membered heteroaryl groups containing 3 or 4 heteroatoms include, without limitation, triazinyl and tetrazinyl, respectively. Exemplary 7-membered heteroaryl groups containing 1 heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Exemplary tricyclic heteroaryl groups include, without limitation, phenanthridinyl, dibenzofuranyl, carbazolyl, acridinyl, phenothiazinyl, phenoxazinyl and phenazinyl.

“Saturated” refers to a ring moiety that does not contain a double or triple bond, i.e., the ring contains all single bonds.

Alkyl, cycloalkyl, heterocyclyl, aryl, and heteroaryl groups may be optionally substituted. Optionally substituted refers to a group which may be substituted or unsubstituted. In general, the term “substituted” means that at least one hydrogen present on a group is replaced with a non-hydrogen substituent, and which upon substitution results in a stable compound, e.g., a compound which does not spontaneously undergo transformation such as by rearrangement, cyclization, elimination, or other reaction. Heteroatoms such as nitrogen, oxygen, and sulfur may have hydrogen substituents and/or non-hydrogen substituents which satisfy the valencies of the heteroatoms and results in the formation of a stable compound.

Exemplary non-hydrogen substituents wherein a moiety is “optionally substituted” as used herein means the moiety may be substituted with any additional moiety selected from, but not limited to, the group consisting of halogen, —CN, —NO₂, —N₃, —SO₂H, —SO₃H, —OH, —OR^{aa}, —N(R^{bb})₂, —N(OR^{cc})R^{bb}, —SH, —SR^{aa}, —C(═O)R^{aa}, —CO₂H, —CHO, —CO₂R^{aa}, —OC(═O)R^{aa}, —OCO₂R^{aa}, —C(═O)N(R^{bb})₂, —OC(═O)N(R^{bb})₂, —NR^{bb}C(═O)R—, —NRbCO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, C(═O)NR^{bb}SO₂R^{aa}, —NR^{bb}SO₂R^{aa}, —SO₂N(R^{bb})₂, —SO₂R″, —S(═O)R^{aa}, —OS(═O)R^{aa}, —B(OR^{cc})₂, C₁₋₁₀ alkyl, C₂₋₁₀ alkenyl, C₂₋₁₀ alkynyl, C₃₋₁₄ carbocyclyl, 3- to 14-membered heterocyclyl, C₆₋₁₄ aryl, and 5- to 14-membered heteroaryl, wherein each alkyl, alkenyl, alkynyl, carbocyclyl, heterocyclyl, aryl, and heteroaryl is independently substituted with 0, 1, 2, 3, 4, or 5 R^{dd} groups, or two geminal hydrogens on a carbon atom are replaced with the group ═O; each instance of R^{aa} is, independently, selected from the group consisting of C₁₋₁₀ alkyl, C₁₋₁₀ perhaloalkyl, C₂₋₁₀ alkenyl, C₂₋₁₀ alkynyl, C₃₋₁₄ carbocyclyl, 3- to 14-membered heterocyclyl, C₆₋₁₄ aryl, and 5- to 14-membered heteroaryl, or two R^{aa} groups are joined to form a 3- to 14-membered heterocyclyl or 5- to 14-membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups; each instance of R^{bb} is, independently, selected from the group consisting of hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —SO₂N(R^{cc})₂, —SOR^{aa}, C₁₋₁₀ alkyl, C₁₋₁₀ perhaloalkyl, C₂₋₁₀ alkenyl, C₂₋₁₀ alkynyl, C₃₋₁₄ carbocyclyl, 3- to 14-membered heterocyclyl, C₆₋₁₄ aryl, and 5- to 14-membered heteroaryl, or two R^{bb} groups are joined to form a 3- to 14-membered heterocyclyl or 5- to 14-membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups; each instance of R^{cc} is, independently, selected from the group consisting of hydrogen, C₁₋₁₀ alkyl, C₁₋₁₀ perhaloalkyl, C₂₋₁₀ alkenyl, C₂₋₁₀ alkynyl, C₃₋₁₄ carbocyclyl, 3- to 14-membered heterocyclyl, C₆₋₁₄ aryl, and 5- to 14-membered heteroaryl, or two R^{cc} groups are joined to form a 3- to 14-membered heterocyclyl or 5- to 14-membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups; and each instance of R^{dd} is, independently, selected from the group consisting of halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(OC₁₋₆ alkyl)(C₁₋₆ alkyl), —N(OH)(C₁₋₆ alkyl), —NH(OH), —SH, —SC₁₋₆ alkyl, —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —B(OH)₂, —B(OC₁₋₆ alkyl)₂, C₁₋₆ alkyl, C₁₋₆ perhaloalkyl, C₂₋₆ alkenyl, C₂₋₆ alkynyl, C₃₋₁₀ carbocyclyl, C₆₋₁₀ aryl, 3- to 10-membered heterocyclyl, and 5- to 10-membered heteroaryl; or two geminal R^{dd} substituents on a carbon atom may be joined to form ═O.

“Halo” or “halogen” refers to fluorine (fluoro, —F), chlorine (chloro, —Cl), bromine (bromo, —Br), or iodine (iodo, —I).

As used herein, the term “composition” is intended to encompass a product comprising the specified ingredients, as well as any product which results, directly or indirectly, from combination of the specified ingredients.

### DETAILED DESCRIPTION OF THE FIGURES

FIG. 1. Rapid Alternating Polarity (rAP) is a new electrolysis mode for synthetic organic electrochemistry. Traditionally, direct current was applied to electrochemical synthesis of organic compounds, where electricity is applied by holding either current (I) or potential (V) at a constant value (FIG. 1, left). Although much less common, alternating current instead of direct current can be applied to electrochemical synthesis as well. Alternating current refers to electric current that periodically changes its direction. For example, the potential change of alternating current follows a sine-wave pattern (FIG. 1, middle) as well as square waveform (FIG. 1, right). Square waveform can be easily accessed by simply alternating the electrode polarity; thus, we refer this waveform as rapid Alternating Polarity (rAP) to distinguish it from other type of AC waveform.

FIG. 2. To demonstrate the effect, the dimerization of 10-undecenoic acid 1 is demonstrated as shown. In fact, dimerization of such a simple carboxylic acid was inoperable under conventional Kolbe electrolysis conditions (entry 1). In fact, this is consistent with the literature report that Kolbe dimerization of this substrate yielded no desired product 2 due to extensive electrode passivation. The experiment with Pt electrodes was repeated in acetone, to no avail (entry 2). Switching electrodes into RVC electrodes has no improvement (entry 3) with DC current. Surprisingly, under the conditions in entry 3, the dimerization proceeded with high efficiency by simply switching the current delivery to rAP, furnishing the desired dimer in 63% yield (entry 4).

FIG. 3. FIG. 3 demonstrates the scope of the rAP-Kolbe dimerization. The functional group tolerance was greatly expanded with this new method, and now various acids containing ester (3), amino (5), free hydroxy (6), ketone (7) and even aryl groups that are susceptible for oxidative degradation (8, 9) afford the corresponding dimer in a good yield. The method can be applied to dimerizing amino acids to synthesize high-value unnatural amino acid without losing chiral information (10 and 11 was obtained as a single diastereomer). Even azetidine 3-carboxylic acid can be dimerized in synthetically useful yield (12).

FIG. 4. Notably, FIG. 4 demonstrates that rAP-cross Kolbe is also possible such that high-value unnatural amino acids can be directly obtained from readily available glutamic acid or aspartic acid in a single step without necessitating any expensive reagent or catalyst.

FIG. 5 demonstrates the scope of the decarboxylative olefination. Decarboxylative olefination proceeded under rAP electrolysis with modified conditions.

FIG. 6 demonstrates examples of the application of the rAP-Kolbe electrolysis products. This method enables access to several compounds that have high commercial value or potential. Moreover, rAP-Kolbe electrolysis could hold great promise from the viewpoint of biomass conversion, since carboxylic acids are ubiquitously found in biomass. In fact, 10-undecenoic acid (starting material for diene 2) is a biomass-derived carboxylic acid. rAP-Kolbe electrolysis has potential for commercial production of sophisticated polymer building blocks from biomass. Additionally, amino acids and derivatives thereof are some of the most versatile chiral building blocks for pharmaceuticals and other chemicals that are able to be produced using the rAP-Kolbe electrolysis methods described herein.

### EXAMPLES

### Common Abbreviations

Commonly used abbreviations include: acetyl (Ac), azo-bis-isobutyrylnitrile (AIBN), atmospheres (Atm), 9-borabicyclo[3.3.1]nonane (9-BBN or BBN), tert-butoxycarbonyl (Boc), di-tert-butyl pyrocarbonate or boc anhydride (BOC₂O), benzyl (Bn), butyl (Bu), Chemical Abstracts Registration Number (CASRN), benzyloxycarbonyl (CBZ or Z), carbonyl diimidazole (CDI), 1,4-diazabicyclo[2.2.2]octane (DABCO), diethylaminosulfur trifluoride (DAST), dibenzylideneacetone (dba), 1,5-diazabicyclo[4.3.0]non-5-ene (DBN), 1,8-diazabicyclo[5.4.0]undec-7-ene (DBU), N,N′-dicyclohexylcarbodiimide (DCC), 1,2-dichloroethane (DCE), dichloromethane (DCM), diethyl azodicarboxylate (DEAD), di-iso-propylazodicarboxylate (DIAD), di-iso-butylaluminumhydride (DIBAL or DIBAL-H), di-iso-propylethylamine (DIPEA), N,N-dimethyl acetamide (DMA), 4-N,N-dimethylaminopyridine (DMAP), N,N-dimethylformamide (DMF), dimethyl sulfoxide (DMSO), 1,1′-bis-(diphenylphosphino)ethane (dppe), 1,1′-bis-(diphenylphosphino)ferrocene (dppf), 1-(3-dimethylaminopropyl)-3-ethylcarbodiimide hydrochloride (EDCI), ethyl (Et), ethyl acetate (EtOAc), ethanol (EtOH), 2-ethoxy-2H-quinoline-1-carboxylic acid ethyl ester (EEDQ), diethyl ether (Et₂O), O-(7-azabenzotriazole-1-yl)-N,N,N′N′-tetramethyluronium hexafluorophosphate acetic acid (HATU), acetic acid (HOAc), 1-N-hydroxybenzotriazole (HOBt), high pressure liquid chromatography (HPLC), iso-propanol (IPA), lithium hexamethyl disilazane (LiHMDS), methanol (MeOH), melting point (mp), MeSO₂—(mesyl or Ms), methyl (Me), acetonitrile (MeCN), m-chloroperbenzoic acid (MCPBA), mass spectrum (ms), methyl t-butyl ether (MTBE), N-bromosuccinimide (NBS), N-carboxyanhydride (NCA), N-chlorosuccinimide (NCS), N-methylmorpholine (NMM), N-methylpyrrolidone (NMP), pyridinium chlorochromate (PCC), pyridinium dichromate (PDC), phenyl (Ph), propyl (Pr), iso-propyl (i-Pr), pounds per square inch (psi), pyridine (pyr), room temperature (rt or RT), tert-butyldimethylsilyl or t-BuMe₂Si (TBDMS), triethylamine (TEA or Et₃N), 2,2,6,6-tetramethylpiperidine 1-oxyl (TEMPO), triflate or CF₃SO₂—(Tf), trifluoroacetic acid (TFA), 1,1′-bis-2,2,6,6-tetramethylheptane-2,6-dione (TMHD), O-benzotriazol-1-yl-N,N,N′,N′-tetramethyluronium tetrafluoroborate (TBTU), thin layer chromatography (TLC), tetrahydrofuran (THF), trimethylsilyl or Me₃Si (TMS), p-toluenesulfonic acid monohydrate (TsOH or pTsOH), 4-Me-C₆H₄SO₂— or tosyl (Ts), N-urethane-N-carboxyanhydride (UNCA). Conventional nomenclature including the prefixes normal (n), iso (i-), secondary (sec-), tertiary (tert-) and neo have their customary meaning when used with an alkyl moiety. (J. Rigaudy and D. P. Klesney, Nomenclature in Organic Chemistry, IUPAC 1979 Pergamon Press, Oxford.).

General Procedure for rAP-Kolbe Electrolysis:

An ElectraSyn vial (5 mL) with a stir bar was charged with carboxylic acid (1.5 mmol, 1 equiv), acetone (3.5 mL) and tetramethyl ammonium hydroxide (referred to as Me₄NOH, or TMAOH, 0.15 mmol, 10 mol %, using 3M aqueous solution prepared in advance). An ElectraSyn vial cap equipped with two electrodes (RVC (Reticulated Vitreous Carbon), 9 mm wide, 40 mm length, 1 mm thickness) was inserted into the mixture. After setting up the vial, the mixture was stirred and degassed via an argon balloon for roughly 1 minute. The reaction mixture was electrolyzed under a rAP conditions (electrolysis condition settings: current: 60 mA, Alternating frequency: 50 ms (10 Hz), amount of charge:8 F/mol). After the rAP electrolysis was completed, the ElectraSyn vial cap was removed and the electrodes were rinsed with AcOEt, which was combined with the crude mixture. The crude mixture was further diluted with AcOEt and aqueous HCl (1N) was then added. The organic layers were concentrated in vacuo. The crude material is a composition that may include different amounts of starting materials and byproducts. Byproducts specifically include alkenyl compounds from decarboxylative olefination and alkanyl compounds from decarboxylative protonation. The crude material was purified by column chromatography, preparative thin layer chromatography (pTLC), or crystallization to furnish the desired product with satisfactory purity.

Examples of rAP-Kolbe Electrolysis

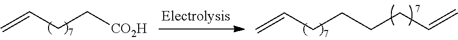

| | | Yield | |
|---|---|---|---|
| Entry | Conditions | (remaining SM) | |
| 1. Conventional Kolbe electrolysis with DC | NaOMe (10 mol %), MeOH, | 0% | (63%) |
| | (+)Pt/(−)Pt, 60 mA, DC, 8 F/mol | | |
| 2. Kolbe electrolysis in acetone | Me₄N•OH (10 mol %), acetone, | 0% | (78%) |
| | (+)Pt/(−)Pt, 60 mA, DC, 8 F/mol | | |
| 3. Kolbe electrolysis with RVC electrode | Me₄N•OH (10 mol %), acetone, | <5% | (>95%) |
| | (+)RVC/(−)RVC, 60 mA, DC, 8 F/mol | | |
| 4. rAP-Kolbe electrolysis | Me₄N•OH (10 mol %), acetone, | 63% | (15%) |
| | 60 mA, RVC/RVC, rAP(50 ms), 8 F/mol | | |

### FIG. 2

### Example (FIG. 2, Entry 4)

This example is conducted with 11-undecenoic acid (1.5 mmol) according to the general procedures. After rAP electrolysis and work-up, the crude mixture was obtained. This crude material contained eicosa-1,19-diene (rAP-Kolbe product) and 1,10-decadiene (alkenyl compound, 4% for rAP-Kolbe product), 1-decene (alkanyl compound, 2% for rAP-Kolbe product). The crude material was purified by column chromatography and eicosa-1,19-diene is obtained in 63% yield. The product is identified by NMR. ¹H NMR (600 MHz, CDCl₃): 5.82 (ddt, J=17.0, 10.0, 6.8 Hz, 2H), 5.00 (ddt, J=17.0, 3.2, 1.6 Hz, 2H), 4.92 (ddt, J=10.0, 3.2, 1.6 Hz, 2H), 2.03 (dq, J=1.6, 6.8 Hz, 4H), 1.43-1.27 (m, 28H). ¹³C NMR (151 MHz, CDCl₃): 139.3, 114.1, 33.8, 29.7, 29.6, 29.5, 29.2, 29.0

### Comparative Example (FIG. 2, Entry 1, 2, 3)

Entry 1, 2, 3 are comparative examples conducted under the described condition in FIG. 2 with DC (direct current). The yields of entries 1-3 are much lower than entry 4 (rAP-Kolbe general conditions).

General Procedure for rAP-Kolbe Electrolysis for Scale Up:

A beaker (250 mL) with a stir bar was charged with carboxylic acid (200 mmol, 1 equiv), acetone (100 mL) and tetramethyl ammonium hydroxide (20 mmol, 10 mol %, using tetramethyl hydroxide pentahydrate). A rubber vial cap equipped with two electrodes (RVC, 20 mm wide, 80 mm length, 10 mm thickness) was inserted into the mixture. After setting up the vial, the mixture was stirred and degassed via an argon balloon for roughly 1 minute. The two electrodes were connected to the electrolysis equipment. (wave generator: RIGOL DG812, Wave amplifier: Accel Instrument TS250). The reaction mixture was electrolyzed under a rAP conditions (electrolysis condition setting: voltage: 10V, wave form: square form alternating, Polarity Alternating frequency: 150 ms 3.33 Hz). After electrolysis was continued for 5 days, the vial cap was removed and electrodes were rinsed with AcOEt, which was combined with the crude mixture. The crude mixture was further diluted with AcOEt and aqueous HCl (1N) was then added. The organic layers were concentrated in vacuo. The crude material was diluted to hexane and washed 1N NaOH 3 times to remove the remained carboxylic acid and obtained the desired product. Further purification by crystallization or column chromatography was carried out as needed.

Reaction Optimization for the rAP-Kolbe Electrolysis (Table 1)

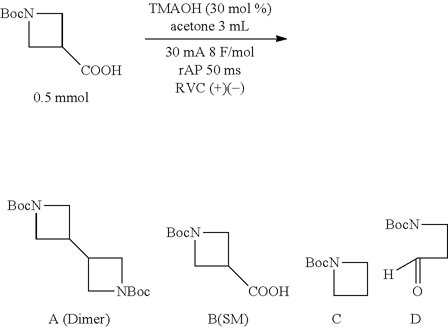

### 1) the 1st Survey (Entries 1-17) at 30 mA and 0.5 Mmol Substrate:

The initial standard conditions are shown in Entry 1 (TMAOH 30 mol %, 30 mA, 8 F/mol, rAP 50 ms).

Various bases are examined and at 30 mA, Entry 9 with KOH 30 mol %, gave the best yield. TMAOH gave a lower yield at 30 mA because of low conversion.

### 2) the Adjusted Conditions are Shown in 2nd Survey (Entry 18, 19) at 60 mA and 0.5 Mmol Substrate:

In the case of changing the current into 60 mA, Entry 19 with TMAOH 10 mol % gave a better yield than Entry 18 with KOH 30 mol %.

### 3) the 3^{rd} Survey (Entry 20-25) at 60 mA and 1.5 Mmol Substrate

Voltage at these reactions are described in Table 1. (voltage on the beginning of the reaction is left and voltage of the end of reaction is right.)

Entry 21 (TMAOH 10 mol %, 60 mA, 1.5 mmol) gave the best yield 36.4% at the acceptable voltage. The conditions for Entry 21 were adapted for the general procedure with the most favorable conditions.

Entry 20 (KOH 10 mol % or NH₄OH 10 mol %) while the yield was also good, the voltage is too high for further scale up.

In the case of adding a support electrolyte (0.01M in acetone solvent) (Entry 23-25), the voltage was decreased, however the yield was also decreased.

Furthermore, the other solvents tested such as AN, MeOH, CH₂Cl₂, DMF, DMSO, GBL, and Propylene Carbonate were examined under the best reaction conditions, but the yields were <10%.

TABLE 1

| | | Voltage | dimer |
|---|---|---|---|
| entry | condition | (V) | % |
| 1 | TMAOH30 mol % | | 7.4 |
| 2 | TMAOH10 mol % | | 8.4 |
| 3 | TMAOH0 mol % | | 1.2 |
| 4 | TMAOH30 mol % 24 F/mol | | 18.7 |
| 5 | TMAOH30 mol % 10 mA | | 0.0 |
| 6 | TMAOH30 mol % 5 ms | | 0.0 |
| 7 | TEAOH 30 mol % | | 1.9 |
| 8 | NaOH 30 mol % | | 2.9 |
| 9 | KOH 30 mol % | | 23.7 |
| 11 | CsOH 30 mol % | | 4.2 |
| 12 | Ca(OH)2 30 mol % | | 0.0 |
| 13 | Ba(OH)2 30 mo % | | 0.0 |
| 14 | LiOH 30 mo % | | 7.3 |
| 15 | KOH 10 mol % | | 8.0 |
| 16 | KOH 100 mol % | | 7.3 |
| 17 | KOH 30 mol % pivOH 30 mol % | | 23.4 |
| 18 | KOH 30 mol % 60 mA | | 23.5 |
| 19 | TMAOH 10 mol % 60 mA | | 27.3 |
| 20 | KOH 10 mol % 60 mA 1.5 mmol | 18.9/>30 | 32.7 |
| 21 | TMAOH10 mol % 60 mA 1.5 mmol | 13.6/18.1 | 36.4 |
| 22 | NH4OH 10 mol % 60 mA 1.5 mmol | 14.9/>30 | 34.0 |
| 23 | TMAOH10 mol % LiClO4 0.01M AT | 10.3/15.1 | 13.9 |
| 24 | TMAOH10 mol % NaClO4 0.01M AT | 9.4/13.2 | 12.5 |
| 25 | TMAOH10 mol % TMABF4 0.01M AT | 12.1/18.8 | 18.8 |

### General Procedure for Decarboxylative Olefination:

An ElectraSyn vial (5 mL) with a stir bar was charged with carboxylic acid (1.5 mmol, 1 equiv), acetone (3.5 mL), and tetramethyl ammonium hydroxide (Me₄NOH, 0.45 mmol, 30 mol % using 3M aqueous solution prepared in advance), and pivalic acid (PivOH, 0.3 mmol using 3M acetone solution prepared in advance). An ElectraSyn vial cap equipped with two electrodes (Graphite, 9 mm wide, 40 mm length) was inserted into the mixture. After setting up the vial, the mixture was stirred and degassed via an argon balloon for roughly 1 minute. The reaction mixture was electrolyzed under an Alternating Polarity condition (electrolysis condition setting: current: 100 mA, Alternating frequency: 5 s, the amount of charge:5 F/mol). After electrolysis was finished, the ElectraSyn vial cap was removed and electrodes were rinsed with AcOEt, which was combined with the crude mixture. The crude mixture was further diluted with AcOEt and aqueous HCl (1N) was then added. The organic layers were concentrated in vacuo. The crude material was purified by column chromatography, preparative thin layer chromatography (pTLC), or crystallization to furnish the desired product.

Examples for the Application of the rAP-Kolbe Product:

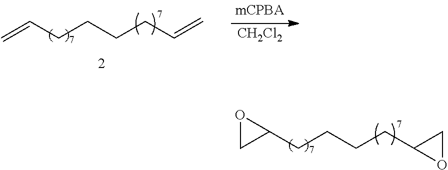

To a solution of diene 2 (10.8 mmol 3.0 g) in CH2Cl2 (50 mL) was added meta-chloroperoxybenzoic acid (mCPBA, 3.0 eq, 32.3 mmol, 7.97 g, 70% purity with water), the solution was stirred for 12 h at room temperature. The mixture was filtered to remove the white solid and washed with water and 1N—NaOH and brine. The organic layer was concentrated and purified by column chromatography. The epoxy compound was obtained as a white solid (91% yield).

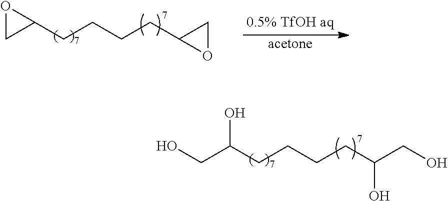

To a solution of epoxy compound (3.22 mmol, 1.0 g) in acetone (20 mL) was added 0.5 wt % aqueous solution of trifluoromethanesulfonic acid (0.5% TfOHaq, 5 mL) at room temperature, the solution was stirred for 12 h at 50° C. The solution was cooled gradually at room temperature and then in ice bath. The resulting white suspension afforded the white solid by filtration. The white solid is washed with acetone and dried in vacuo. The titled compound was obtained as a white solid (82% yield)

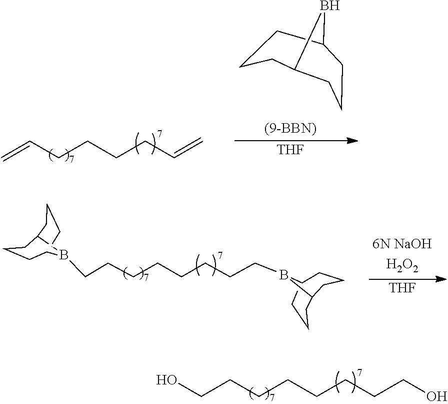

To a solution of diene 2 (1.08 mmol, 0.3 g) in THE (3 mL) was added 9-BBN(9-borabicyclo[3.3.1]nonane) of 0.5M THF solution (4.0 eq, 4.32 mmol, 8.6 mL) at 0° C., the solution was stirred for 2 h at 40° C. To the resulting mixture of 9-BBN addition product was added 6N NaOH aq. (2 mL) and 30 wt % H₂O₂ aq. (4 mL) at 0° C. The mixture was stirred at 0° C. for 1 h and diluted by AcOEt and water. The organic layer was extracted and washed by water and Na₂S₂O₃ aq. and 1N—HCl and Brine. The organic layer was concentrated and purified by column chromatography. The diol compound was obtained as a white solid (85% yield)

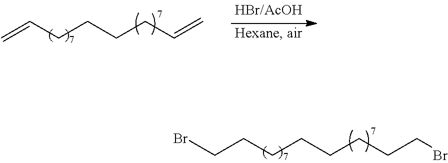

A solution of diene 2 (3.59 mmol, 1.0 g) in hexane (10 mL) was bubbled by air for 5 minutes. To the solution was added HBr 30% solution in AcOH (4.0 eq, 14.4 mmol, 3.88 g) at 0° C., the solution was stirred for 2 h at 0° C. in air atmosphere and diluted by hexane and water. The organic layer was extracted and washed by 1N—NaOH and Brine. The organic layer was concentrated and purified by column chromatography. The dibromo compound was obtained as a white solid (96% yield)

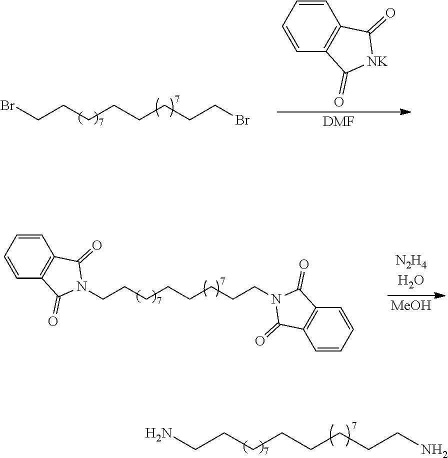

To a solution of dibromo compound (0.45 mmol, 200 mg) in DMF (2 mL) was added potassium phthalimide (2.2 eq, 1.0 mmol, 185 mg) at room temperature. The mixture was stirred for 2 h at 60° C. The resulting mixture was diluted by AcOEt and water. The organic layer was extracted and concentrated in vacuo. The product of phthalimide addition is obtained as white solid. To the solution of the phthalimide product in MeOH (3 mL) was added hydrazine hydrate (20 eq, 9.1 mmol, 0.45 g). The solution was stirred for 12 h at reflux temperature. The resulting solution is cooled in ice bath and changed into a white suspension. The white solid was obtained by filtration and washed by acetone and dried in vacuo. The diamine compound was obtained as a white solid (62% yield)

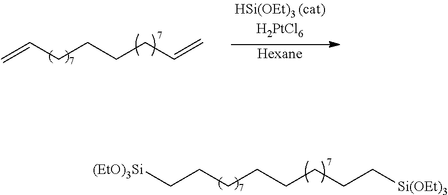

To a solution of diene 2 (3.59 mmol, 1.0 g) and triethoxysilane (2 eq, 7.2 mmol, 1.2 g) in hexane (2 mL) was added chloroplatinic acid hydrate (1 wt % for diene, ca.10 mg), the mixture was stirred for 8 h at 80° C. The resulting mixture was diluted by hexane and filtered. The solution was washed by water and brine. The organic layer was concentrated and purified by column chromatography. The silyl compound was obtained as a liquid (52% yield).

The foregoing disclosure has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the disclosure should, therefore, be determined not with reference to the above description, but should instead be determined with reference to the following appended claims, along with the full scope of equivalents to which such claims are entitled.

This application refers to various issued patents, published patent applications, journal articles, and other publications, each of which are incorporated herein by reference.

What is claimed is:

1. A method of decarboxylative coupling of carboxylic acids, wherein a carboxylic acid substrate is subjected to rapid Alternating Polarity (rAP)-Kolbe electrolysis.

2. The method of claim 1, wherein the rAP-Kolbe electrolysis occurs according to either reaction scheme (i) or reaction scheme (ii): 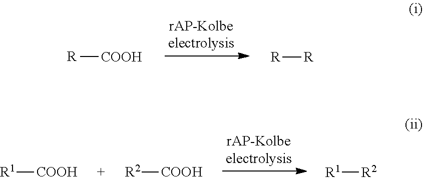
wherein each of R, R¹, and R² is independently selected from H, saturated or unsaturated acyclic or cyclic aliphatic groups, each optionally including one or more heteroatoms and each optionally substituted with alkyl, alkenyl, ester, amino, halo, hydroxy, keto, formyl, aryl, heteroaryl, cycloalkyl or heterocyclic moieties, and aromatic or heteroaromatic ring systems, each optionally substituted with alkyl, alkenyl, ester, amino, halo, hydroxy, keto, formyl, aryl, heteroaryl, cycloalkyl or heterocyclic moieties.

3. The method of decarboxylative coupling of carboxylic acids of either claim 1 or claim 2, wherein the alternating frequency is 1 MHz-0.01 Hz.

4. The method of decarboxylative coupling of carboxylic acids of any one of claims 1-3, wherein the electrodes are composed of Pt, Pd, Ni, Rh, Ti, Pb, conductive metal oxides, or a carbon-based material with surface modification or coating.

5. The method of decarboxylative coupling of carboxylic acids of any one of claims 1-4, wherein the carboxylic acid substrate contains 2-100 carbon atoms.

6. The method of decarboxylative coupling of carboxylic acids of any one of claims 1-5, wherein the carboxylic acid substrate contains heteroatoms.

7. The method of decarboxylative coupling of carboxylic acids of any one of claims 1-6, wherein the carboxylic acid substrate is unsaturated, partially unsaturated, or aromatic.

8. The method of decarboxylative coupling of carboxylic acids of any one of claims 1-7, wherein the carboxylic acid substrate contains a C—N bond.

9. The method of decarboxylative coupling of carboxylic acids of any one of claims 1-8, wherein the carboxylic acid substrate contains alkenyl, ester, amino, free hydroxy, cycloalkyl, ketone, aryl, or heterocycloalkyl moieties.

10. The method of decarboxylative coupling of carboxylic acids of any one of claims 1-9, wherein a supporting electrolyte is added.

11. The method of decarboxylative coupling of carboxylic acids of any one of claims 1-10, wherein the supporting electrolyte is an ammonium salt.

12. The method of decarboxylative coupling of carboxylic acids of any one of claims 1-11, wherein the decarboxylative coupling of carboxylic acids occurs in the presence of a base.

13. The method of decarboxylative coupling of carboxylic acids of claim 12, wherein the amount of base is approximately 0.01 mol % to approximately 200 mol % to the overall amount of the carboxylic acid substrate.

14. The method of decarboxylative coupling of carboxylic acids of claim 13, wherein the amount of the base is approximately 10 to approximately 30 mol % to the overall amount of the carboxylic acid substrate.

15. The method of decarboxylative coupling of carboxylic acids of any one of claims 12-14, wherein the base is NR₄OH, NH₄OH, NaOH, or KOH and R is (C₁-C₆)alkyl.

16. The method of decarboxylative coupling of carboxylic acids of claim 15, wherein the base is NR₄OH and R is (C₁-C₆)alkyl.

17. The method of decarboxylative coupling of carboxylic acids of claim 16, wherein the amount of NR₄OH is approximately 10 mol % to the overall amount of the carboxylic acid substrate.

18. The method of decarboxylative coupling of carboxylic acids of any one of claims 1-17, wherein the solvent has a relative dielectric constant of more than 5.

19. The method of decarboxylative coupling of carboxylic acids of any one of claims 1-18, wherein the solvent contains a carbonyl moiety.

20. The method of decarboxylative coupling of carboxylic acids of claim 19, wherein the carbonyl moiety is a (C₃-C₁₅)ketone.

21. The method of decarboxylative coupling of carboxylic acids of claim 20, wherein the (C₃-C₁₅)ketone is acetone.

22. The method of decarboxylative coupling of carboxylic acids of any one of claims 1-21, wherein the alternating frequency is approximately 10 Hz.

23. The method of decarboxylative coupling of carboxylic acids of any one of claims 1-22, wherein the current is set at 1-1000 mA/mmol.

24. The method of decarboxylative coupling of carboxylic acids of claim 23, wherein the current is set at approximately 60 mA/mmol.

25. The method of decarboxylative coupling of carboxylic acids of any one of claims 1-24, wherein the amount of charge is set at 1-20 F/mol.

26. The method of decarboxylative coupling of carboxylic acids of claim 25, wherein the amount of charge is set at approximately 8-10 F/mol.

27. The method of decarboxylative coupling of carboxylic acids of any one of claims 1-26, wherein the electrodes are RVC electrodes.

28. The method of decarboxylative coupling of carboxylic acids of any one of claims 1-27, wherein the carboxylic acid substrate comprises one carboxylic acid or two carboxylic acids.

29. The method of decarboxylative coupling of carboxylic acids of claim 28, wherein the carboxylic acid substrate comprises one carboxylic acid.

30. The method of decarboxylative coupling of carboxylic acids of any one of claims 1-29, wherein the rAP-Kolbe electrolysis occurs according to the following reaction scheme: 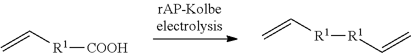
wherein R¹ is (C₁-C₂₀)alkyl or cycloalkyl, optionally substituted with aryl, heteroaryl, O, N, or halo.

31. The method of decarboxylative coupling of carboxylic acids of any one of claims 1-29, wherein the rAP-Kolbe electrolysis occurs according to the following reaction scheme: 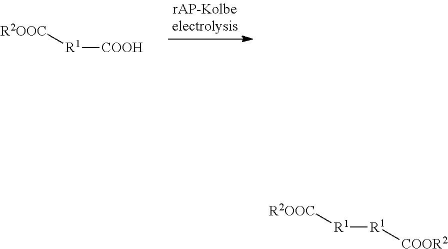
wherein R¹ is (C₁-C₂₀)alkyl or cycloalkyl, optionally substituted with aryl, heteroaryl, O, N, or halo; and
R² is (C₁-C₆)alkyl or cycloalkyl.

32. The method of decarboxylative coupling of carboxylic acids of any one of claims 1-29, wherein the rAP-Kolbe electrolysis occurs according to the following reaction scheme: 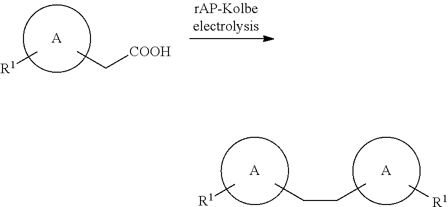
wherein A is (C₃-C₂₀)cycloalkyl; and
R¹ is (C₁-C₂₀)alkyl or (C₃-C₂₀)cycloalkyl, optionally substituted with aryl, heteroaryl, O, N, or halo.

33. The method of decarboxylative coupling of carboxylic acids of any one of claims 1-29, wherein the rAP-Kolbe electrolysis occurs according to the following reaction scheme: 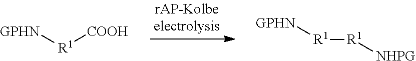
wherein R¹ is (C₁-C₂₀)alkyl, cycloalkyl, or alkyl, optionally substituted with aryl, heteroaryl, O, N, or halo; and
PG is a protecting group including, but not limited to Boc and Cbz.

34. The method of decarboxylative coupling of carboxylic acids of any one of claims 1-29, wherein the rAP-Kolbe electrolysis occurs according to the following reaction scheme: 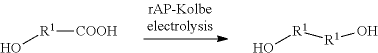
wherein R¹ is (C₁-C₂₀)alkyl, cycloalkyl, or alkyl, optionally substituted with aryl, heteroaryl, O, N, or halo.

35. The method of decarboxylative coupling of carboxylic acids of any one of claims 1-29, wherein the rAP-Kolbe electrolysis occurs according to the following reaction scheme: 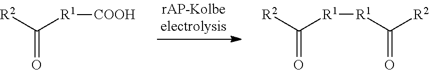
R¹ is (C₁-C₂₀)alkyl or cycloalkyl, optionally substituted with aryl, heteroaryl, O, N, or halo; and
R² is (C₁-C₆)alkyl or cycloalkyl.

36. The method of decarboxylative coupling of carboxylic acids of any one of claims 1-29, wherein the rAP-Kolbe electrolysis occurs according to the following reaction scheme: 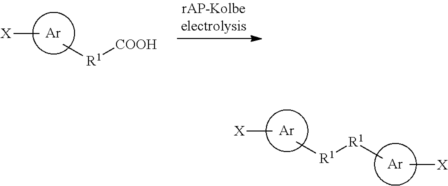
wherein X is halo or OR²;
Ar is aryl, including, but not limited to, Ph and naphthyl;
R¹ is (C₁-C₂₀)alkyl or cycloalkyl, optionally substituted with aryl, heteroaryl, O, N, or halo; and
R² is (C₁-C₆)alkyl or cycloalkyl.

37. The method of decarboxylative coupling of carboxylic acids of any one of claims 1-29, wherein the rAP-Kolbe electrolysis occurs according to the following reaction scheme: 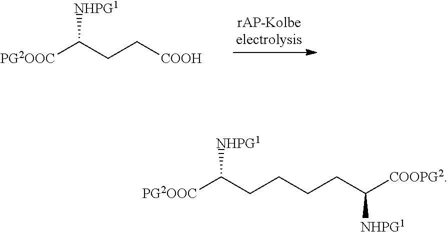
wherein PG¹ is an amino protecting group, including, but not limited to, Boc and Cbz; and
PG² is a carboxylic acid protecting group, including, but not limited to, tBu, Me, and Bn.

38. The method of decarboxylative coupling of carboxylic acids of any one of claims 1-29, wherein the rAP-Kolbe electrolysis occurs according to the following reaction scheme: 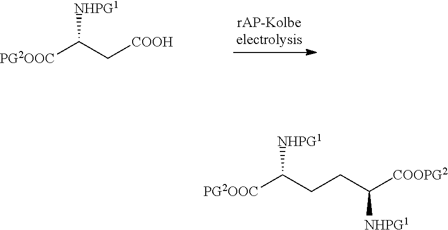
wherein PG¹ is an amino protecting group, including, but not limited to, Boc and Cbz; and
PG² is a carboxylic acid protecting group, including, but not limited to, tBu, Me, and Bn.

39. The method of decarboxylative coupling of carboxylic acids of any one of claims 1-29, wherein the rAP-Kolbe electrolysis occurs according to the following reaction scheme: 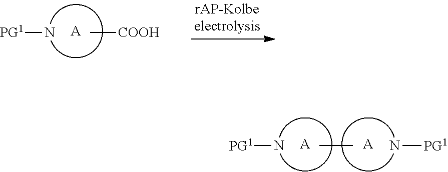
wherein A is (C₃-C₁₀)heterocycle;
PG¹ is an amino protecting group, including, but not limited to, Boc and Cbz.

40. The method of decarboxylative coupling of carboxylic acids of any one of claims 1-39, wherein the carboxylic acid substrate concentration is approximately 1.5 mmol.

41. The method of decarboxylative coupling of carboxylic acids of any one of claims 1-40, wherein the carboxylic acid substrate is treated with approximately 10 mol % NH₄OH.

42. The method of decarboxylative coupling of carboxylic acids of any one of claims 1-40, wherein the solvent is acetone.

43. The method of decarboxylative coupling of carboxylic acids of any one of claims 40-42, wherein the current is set at approximately 60 mA.

44. The method of decarboxylative coupling of carboxylic acids of any one of claims 40-43, wherein the alternating frequency is set at approximately 50 ms.

45. The method of decarboxylative coupling of carboxylic acids of any one of claims 40-44, wherein the amount of charge is set at approximately 8 F/mol.

46. The method of decarboxylative coupling of carboxylic acids of any one of claims 40-45, wherein the electrodes are RVC electrodes.

47. The method of decarboxylative coupling of carboxylic acids of any one of claims 1-39, wherein the carboxylic acid substrate concentration is approximately 200 mmol.

48. The method of decarboxylative coupling of carboxylic acids of claim 47, wherein the carboxylic acid substrate is treated with approximately 10 mol % NH₄OH.

49. The method of decarboxylative coupling of carboxylic acids of either claim 46 or claim 47, wherein the solvent is acetone.

50. The method of decarboxylative coupling of carboxylic acids of any one of claims 47-49, wherein the current is set at approximately 60 mA.

51. The method of decarboxylative coupling of carboxylic acids of any one of claims 47-50, wherein the alternating frequency is set at approximately 150 ms.

52. The method of decarboxylative coupling of carboxylic acids of any one of claims 47-51, wherein the amount of charge is set at approximately 8 F/mol.

53. The method of decarboxylative coupling of carboxylic acids of any one of claims 47-52, wherein the electrodes are RVC electrodes.

54. The method of decarboxylative coupling of carboxylic acids of any one of claims 1-28, wherein the carboxylic acid substrate comprises two carboxylic acids.

55. The method of decarboxylative coupling of carboxylic acids of claim 54, wherein one or both of the two different carboxylic acids is an amino acid or derivative thereof.

56. The method of decarboxylative coupling of carboxylic acids of claim 54, wherein the rAP-Kolbe electrolysis occurs according to the following reaction scheme: 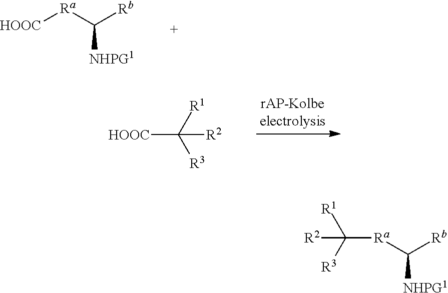
wherein each of R^{a} and R^{b} is independently selected from single bond or H or (C₁-C₂₀)alkyl or cycloalkyl, optionally substituted with aryl, heteroaryl, O, N, or halo; and
PG¹ is an amino protecting group, including, but not limited to, Boc and Cbz.

57. The method of decarboxylative coupling of carboxylic acids of claim 54, wherein the rAP-Kolbe electrolysis occurs according to the following reaction scheme: 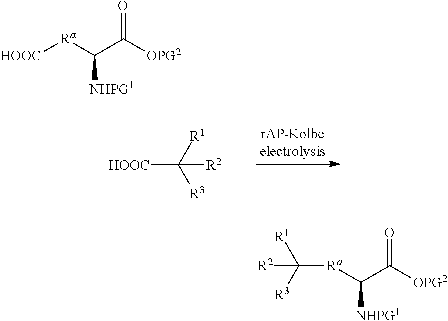
wherein R^{a} is single bond or (C₁-C₂₀)alkyl, cycloalkyl, optionally substituted with aryl, heteroaryl, O, N, or halo;
PG¹ is an amino protecting group, including, but not limited to, Boc and Cbz; and
PG² is a carboxylic acid protecting group, including, but not limited to, tBu, Me, and Bn.

58. The method of decarboxylative coupling of carboxylic acids of claim 54, wherein the rAP-Kolbe electrolysis occurs according to the following reaction scheme: 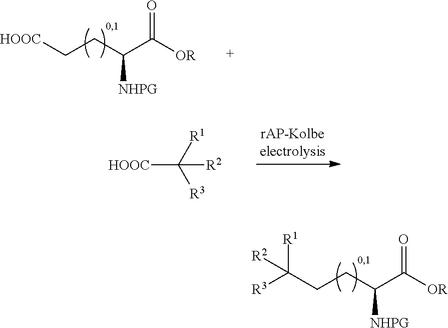
wherein PG is an amino protecting group, including, but not limited to, Boc and Cbz;
R is (C₁-C₆)alkyl or (C₁-C₆)alkylaryl; and
each of R¹, R², and R³ is independently selected from H, (C₁-C₁₂)alkyl, (C₁-C₁₂)haloalkyl, (C₂-C₁₂)alkenyl, (C₁-C₆)alkyl ester, and (C₁-C₆)alkyl-NHBoc.

59. The method of decarboxylative coupling of carboxylic acids of any one of claims 54-58, wherein the carboxylic acid substrate concentration is approximately 1.5 mmol.

60. The method of decarboxylative coupling of carboxylic acids of any one of claims 54-59, wherein the carboxylic acid substrate is treated with approximately 10 mol % NMe₄OH.

61. The method of decarboxylative coupling of carboxylic acids of any one of claims 54-60, wherein the carboxylic acid substrate is treated with approximately 2 equivalents of NMe₄BF₄.

62. The method of decarboxylative coupling of carboxylic acids of any one of claims 54-61, wherein the solvent is acetone.

63. The method of decarboxylative coupling of carboxylic acids of any one of claims 54-62, wherein the current is set at approximately 60 mA.

64. The method of decarboxylative coupling of carboxylic acids of any one of claims 54-63, wherein the alternating frequency is set at approximately 50 ms.

65. The method of decarboxylative coupling of carboxylic acids of any one of claims 54-64, wherein the amount of charge is set at approximately 10 F/mol.

66. The method of decarboxylative coupling of carboxylic acids of any one of claims 54-65, wherein the electrodes are RVC electrodes.

67. A method of decarboxylative olefination of carboxylic acids, wherein the carboxylic acid substrate is subjected to rAP electrolysis.

68. The method of decarboxylative olefination of carboxylic acids of claim 67, wherein the rAP electrolysis occurs according to either reaction scheme (i) or reaction scheme (ii): 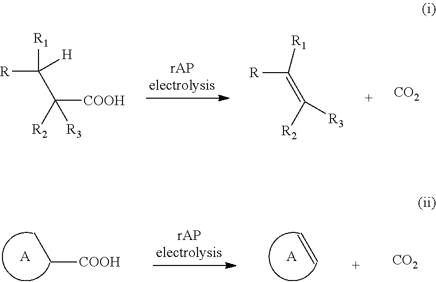
wherein R¹, R² and R³ are optionally H or substituted (C₁-C₂₀)alkyl, (C₂-C₂₀)alkenyl, (C₁-C₂₀)hydroxyalkyl, and N-protected (C₁-C₂₀)aminoalkyl; and
A is optionally substituted, saturated or partially unsaturated, (C₃-C₈)cycloalkyl or (C₃-C₈)heterocycloalkyl.

69. The method of decarboxylative olefination of carboxylic acids of either claim 67 or claim 68, wherein the concentration of the carboxylic acid substrate is approximately 1.5 mmol.

70. The method of decarboxylative olefination of carboxylic acids of any one of claims 67-69, wherein the carboxylic acid substrate is treated with approximately 30 mol % tetramethyl ammonium hydroxide.

71. The method of decarboxylative olefination of carboxylic acids of any one of claims 67-70, wherein the carboxylic acid substrate is treated with approximately 20 mol % pivOH.

72. The method of decarboxylative olefination of carboxylic acids of any one of claims 67-71, wherein the solvent is acetone.

73. The method of decarboxylative olefination of carboxylic acids of any one of claims 67-72, wherein the current is set at approximately 100 mA.

74. The method of decarboxylative olefination of carboxylic acids of any one of claims 67-73, wherein the alternating frequency is set at approximately 5 s.

75. The method of decarboxylative olefination of carboxylic acids of any one of claims 67-74, wherein the amount of charge is set at 5 F/mol.

76. The method of decarboxylative olefination of carboxylic acids of any one of claims 67-75, wherein the electrodes are Graphite electrodes.

77. The method of decarboxylative olefination of carboxylic acids of any one of claims 67-76, wherein the rAP electrolysis occurs according to the following reaction scheme: 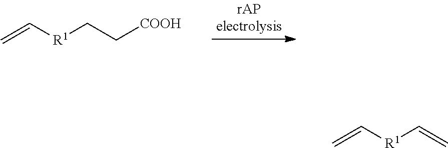
wherein R¹ is (C₁-C₂₀)alkyl or cycloalkyl, optionally substituted with aryl, heteroaryl, O, N, or halo.

78. The method of decarboxylative olefination of carboxylic acids of any one of claims 67-77, wherein the rAP electrolysis occurs according to the following reaction scheme:

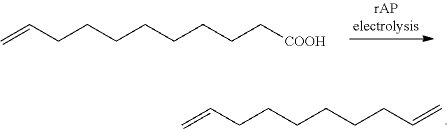

79. The method of decarboxylative olefination of carboxylic acids of any one of claims 67-76, wherein the rAP-Kolbe electrolysis occurs according to the following reaction scheme: 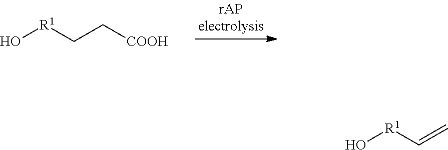
wherein R¹ is (C₁-C₂₀)alkyl or cycloalkyl, optionally substituted with aryl, heteroaryl, O, N, or halo.

80. The method of decarboxylative olefination of carboxylic acids of any one of claims 67-76 and 79, wherein the rAP-Kolbe electrolysis occurs according to the following reaction scheme:

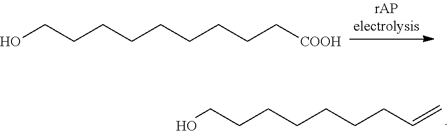

81. The method of decarboxylative olefination of carboxylic acids of any one of claims 67-76, wherein the rAP-Kolbe electrolysis occurs according to the following reaction scheme: 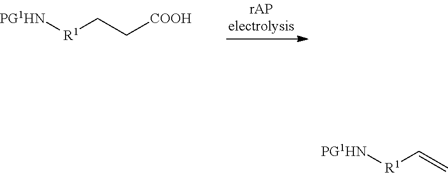
R¹ is (C₁-C₂₀)alkyl or cycloalkyl, optionally substituted with aryl, heteroaryl, O, N, or halo; and
PG¹ is an amino protecting group, including, but not limited to, Boc and Cbz.

82. The method of decarboxylative olefination of carboxylic acids of any one of claims 67-76 and 81, wherein the rAP-Kolbe electrolysis occurs according to the following reaction scheme:

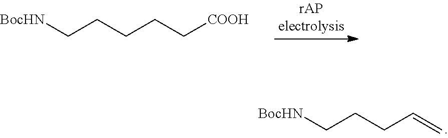 [SMILES: CC(C)(C)OC(=O)NCCCCCC(=O)O ; C=CCCCNC(=O)OC(C)(C)C]

83. The method of decarboxylative olefination of carboxylic acids of any one of claims 67-76, wherein the rAP-Kolbe electrolysis occurs according to the following reaction scheme: 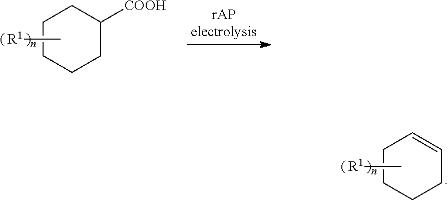
each R¹ is independently (C₁-C₂₀)alkyl or cycloalkyl, optionally substituted with aryl, heteroaryl, O, N, or halo;
or two R¹ together form (C₄-C₁₂)cycloalkyl, optionally substituted with (C₁-C₂₀)alkyl, cycloalkyl, aryl, heteroaryl, O, N, or halo; and
n is 0, 1, or 2.

84. A composition obtained by the decarboxylative coupling of carboxylic acids of any one of claims 1-53, wherein the composition is formed according to the following reaction: 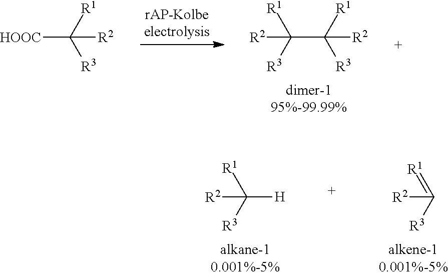
wherein R¹, R², and R³ are each independently C₁-C₅₀ alkyl, optionally substituted with heteroatoms.

85. A composition obtained by the decarboxylative coupling of carboxylic acids of any one of claims 54-66, wherein the composition is formed according to the following reaction: 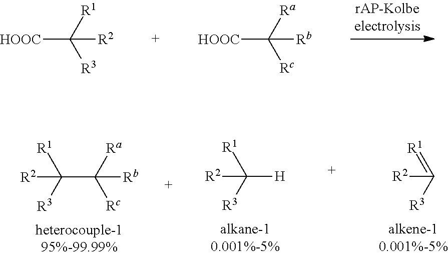
wherein R¹, R², R³, R^{a}, R^{b}, and R^{c} are each independently C₁-C₅₀ alkyl, optionally substituted with heteroatoms.

86. Any 1) method of decarboxylative coupling of a carboxylic acid substrate, wherein the carboxylic acid substrate comprises either one carboxylic acid or two carboxylic acids and subjected to rAP-Kolbe electrolysis; 2) method of decarboxylative olefination of a carboxylic acid substrate, wherein the carboxylic acid substrate is subjected to rAP electrolysis; 3) composition obtained by decarboxylative coupling of carboxylic acids; or 4) any method of preparing chemicals including monomers, polymers, additives, adhesives, solvents, pharmaceuticals, amino acids and derivatives thereof, agricultural chemicals, fragrances, fuels, lubricants, or electronic materials according to the above methods, as disclosed herein.
